SEQUENCE LISTING (41 sequences)

SEQ ID NO: 1 - DNA, 432 bp, Homo sapiens
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactag

SEQ ID NO: 2 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 3 - protein, 10 aa, artificial sequence
  MDK 12-21 peptide
LALLALTSAV

SEQ ID NO: 4 - protein, 9 aa, artificial sequence
  MDK 13-21 peptide
ALLALTSAV

SEQ ID NO: 5 - protein, 10 aa, artificial sequence
  MDK 13-22 peptide
ALLALTSAVA

SEQ ID NO: 6 - protein, 9 aa, artificial sequence
  MDK 14-22 peptide
LLALTSAVA

SEQ ID NO: 7 - protein, 10 aa, artificial sequence
  MDK 113-122 peptide
NAQCQETIRV

SEQ ID NO: 8 - protein, 9 aa, artificial sequence
  MDK 114-122 peptid
AQCQETIRV

SEQ ID NO: 9 - protein, 15 aa, artificial sequence
  MDK 1-15 peptide
MQHRGFLLLTLLALL

SEQ ID NO: 10 - protein, 15 aa, artificial sequence
  MDK 4-18 peptide
RGFLLLTLLALLALT

SEQ ID NO: 11 - protein, 13 aa, artificial sequence
  MDK 9-21 peptide
LTLLALLALTSAV

SEQ ID NO: 12 - protein, 14 aa, artificial sequence
  MDK 9-22 peptide
LTLLALLALTSAVA

SEQ ID NO: 13 - protein, 15 aa, artificial sequence
  MDK 9-23 peptide
LTLLALLALTSAVAK

SEQ ID NO: 14 - protein, 15 aa, artificial sequence
  MDK 14-28 peptide
LLALTSAVAKKKDKV

SEQ ID NO: 15 - protein, 15 aa, artificial sequence
  MDK 110-124 peptide
ARYNAQCQETIRVTK

SEQ ID NO: 16 - protein, 11 aa, artificial sequence
  PADRE PEPTIDE
KXVAAWTLKAA

SEQ ID NO: 17 - protein, 10 aa, artificial sequence
  MDK 63-72 peptide
AQTQRIRCRV

SEQ ID NO: 18 - protein, 15 aa, artificial sequence
  MDK 18-32 peptide
TSAVAKKKDKVKKGG

SEQ ID NO: 19 - protein, 15 aa, artificial sequence
  MDK 25-39 peptide
KDKVKKGGPGSECAE

SEQ ID NO: 20 - protein, 15 aa, artificial sequence
  MDK 38-52 peptide
AEWAWGPCTPSSKDC

SEQ ID NO: 21 - protein, 13 aa, artificial sequence
  MDK 52-64 peptide
CGVGFREGTCGAQ

SEQ ID NO: 22 - protein, 15 aa, artificial sequence
  MDK 64-78 peptide
QTQRIRCRVPCNWKK

SEQ ID NO: 23 - protein, 15 aa, artificial sequence
  MDK 70-84 peptide
CRVPCNWKKEFGADC

SEQ ID NO: 24 - protein, 15 aa, artificial sequence
  MDK 74-88 peptide
CNWKKEFGADCKYKF

SEQ ID NO: 25 - protein, 15 aa, artificial sequence
  MDK 78-92 peptide
KEFGADCKYKFENWG

SEQ ID NO: 26 - protein, 15 aa, artificial sequence
  MDK 84-98 peptide
CKYKFENWGACDGGT

SEQ ID NO: 27 - protein, 15 aa, artificial sequence
  MDK 89-103 peptide
ENWGACDGGTGTKVR

SEQ ID NO: 28 - protein, 15 aa, artificial sequence
  MDK 99-113 peptide
GTKVRQGTLKKARYN

SEQ ID NO: 29 - protein, 15 aa, artificial sequence
  MDK 105-119 peptide
GTLKKARYNAQCQET

SEQ ID NO: 30 - protein, 15 aa, artificial sequence
  MDK 119-133 peptide
ETIRVTKPCTPKTKA

SEQ ID NO: 31 - protein, 13 aa, artificial sequence
  HA 306-318 peptide
PKYVKQNTLKLAT

SEQ ID NO: 32 - protein, 15 aa, artificial sequence
  A3 152-166 peptide
EAEQLRAYLDGTGVE

SEQ ID NO: 33 - protein, 15 aa, artificial sequence
  MT 2-16 peptide
AKTIAYDEEARRGLE

SEQ ID NO: 34 - protein, 16 aa, artificial sequence
  B1 21-36 peptide
TERVRLVTRHIYNREE

SEQ ID NO: 35 - protein, 13 aa, artificial sequence
  YKL peptide
AAYAAAKAAALAA

SEQ ID NO: 36 - protein, 20 aa, artificial sequence
  LOL 191-210 peptide
ESWGAVWRIDTPDKLTGPFT

SEQ ID NO: 37 - protein, 17 aa, artificial sequence [sequence omitted]

SEQ ID NO: 38 - protein, 14 aa, artificial seqeunce
  E2/E168 peptide
AGDLLAIETDKATI

SEQ ID NO: 39 - protein, 9 aa, artificial seqeunce
  survivine 96-104 peptide
LTLGEFLKL

SEQ ID NO: 40 - protein, 10 aa, artificial sequence
  survivine 95-104 peptide
ELTLGEFLKL

SEQ ID NO: 41 - protein, 9 aa, artificial sequence
  survivine 2B 80-88 peptide
AYACNTSTL

(19) United States
(12) Patent Application Publication
(10) Pub. No.: US 2011/0159022 A1
(43) Pub. Date: Jun. 30, 2011

(54) Immunogenic Peptides Derived from the Midkine Protein, as an Anticancer Vaccine

(76) Inventors: Jérôme Kerzerho, Chatou (FR); Bernard Maillere, Versailles (FR); Emmanuel Favry, Savigny Sur Orge (FR)

(21) Appl. No.: 13/000,302

(22) PCT Filed: Jun. 20, 2009
(86) PCT No.: PCT/FR09/00744

§ 371 Date: Mar. 21, 2011

(30) Foreign Application Priority Data
Jun. 20, 2008 (FR) 0803466

(51) Int. Cl. A61K 39/00; C07K 2/00; C07K 7/06; C07K 7/08; C07K 14/00; C12N 15/12; C12N 15/63; C12Q 1/02; A61P 35/00; A61P 37/04

(52) U.S. Cl. 424/185.1; 530/300; 530/328; 530/326; 530/327; 530/402; 536/23.5; 435/320.1; 435/29

(57) ABSTRACT

A peptide derived from the Midkine protein, comprising at least one CD4⁺ T or CD8⁺ T epitope restricted by the HLA molecules predominant in the Caucasian population, or a polynucleotide encoding said peptide, as an anticancer vaccine or as a reagent for immunomonitoring of the cellular response against Midkine over the course of a cancer or of an anticancer treatment.

The invention relates to the use, as an anticancer vaccine, of peptides derived from the midkine protein which are capable of inducing CD4⁺ T and/or CD8⁺ T lymphocytes that recognize said midkine protein in the majority of individuals of the caucasian population, in many types of cancers.

The invention also relates to the use of such peptides recognized by CD4⁺ T and/or CD8⁺ T lymphocytes specific for the midkine protein, in the majority of individuals of the caucasian population, in many types of cancers, as a reagent for immunomonitoring of the cellular response against midkine over the course of a cancer or of an anticancer treatment.

Tumor cells express a collection of proteins which healthy cells do not express, or express very little, or which are found only in a few cell types. These proteins which are preferentially expressed in tumor cells can constitute a tumor antigen, i.e. a protein present in the tumor and which induces an immune response capable of recognizing the tumors and, ideally, of eliminating them. This response may be both an antibody response, insofar as the antigen is a membrane antigen, and a cellular response involving CD8⁺ or CD4⁺ T lymphocytes. Most tumor antigens are intracellular and induce a cellular response. They constitute preferred targets for the development of vaccines.

T lymphocytes contribute to the cellular immune response directed against tumors. They can be induced spontaneously in patients suffering from cancer and infiltrate tumors, giving spontaneous regression in rare cases. They can be induced by vaccines which are planned so as to facilitate their recruitment. They are two types of T lymphocytes involved in antitumor immunity. CD8⁺ T lymphocytes are cytotoxic (CD8⁺ CTLs) and can lyse tumor cells. The lysis of cells during their recognition involves perforin and granzymes. CD8⁺ T lymphocytes recognize the tumor antigen in the form of peptides, called CD8⁺ T epitopes, which are presented to them by the class I HLA molecules (HLA-A, HLA-B and HLA-C) present at the surface of tumors. Helper CD4⁺ T lymphocytes recognize tumor antigens in the form of peptides, called CD4⁺ T epitopes, which are presented to them by the class II HLA molecules. The recognition of tumors by CD4⁺ T lymphocytes can occur directly when the tumors express class II molecules or indirectly through the uptake of cell debris by dendritic cells, which are cells that have a high number of class II HLA molecules at their surface. The CD4⁺ T lymphocytes involved in antitumor immunity play a multiple role in the control of tumors and are in particular involved in recruiting and maintaining CD8⁺ CTLs. CD4⁺ T lymphocytes play a role in activating dendritic cells (DCs) via a CD40-dependent mechanism. They increase IL-12 secretion by DCs and the expression, at their surface, of costimulatory molecules or adhesion molecules (I-CAM-1, CD80, CD86). This activation allows the recruitment of CTLs. In addition, the results observed in mice which do not express class I molecules also indicate that helper T lymphocytes exert tumor control via CTL-independent mechanisms, probably via macrophage activation. Finally, CD4⁺ T lymphocytes can themselves be cytotoxic. Dendritic cells are also involved in antitumor immunity by initiating this response. The naïve tumor-specific T lymphocytes are in fact recruited and activated by dendritic cells and not by the tumor cells.

The discovery of the first tumor antigens in the 1990s was responsible for many studies on these proteins expressed in tumors. Tumor antigens have been divided up into several categories according to their mode of expression.

Tumor-Specific Antigens

This is the largest group of antigens, which was initially discovered in melanomas, but which is in fact expressed in many tumors. These antigens are also called “Cancer Testis” owing to their expression in the testicles, which is the only healthy tissue which expresses them. Some of these antigens are also expressed in the placenta or the ovaries. Since the testicles and the placenta are devoid of conventional HLA molecules, these antigens are not visible to the T lymphocytes in the healthy tissues. The main antigens are the MAGE-A, MAGE-B, MAGE-C, GAGE, LAGE and SSX antigens.

Differential Antigens

The differential antigens are proteins expressed by tumors and by the cell tissue which gave rise to the tumor. The most well-known examples are melanoma antigens, which are also expressed in melanocytes. They are the tyrosinases (TYRO, TRP-1 and TRP-2) and the Gp100 and MELAN-A/MART-1 antigens. Other differential antigens are also known for prostate tumors (kallikrein-4 and PSA) or cancer of the digestive tract (CEA).

Overexpressed Antigens

Overexpressed antigens are proteins that are highly expressed in many tumor cells, although their level of expression is not very high in normal cells. This is the case of the HER-2/neu antigen which is found in approximately 30% of breast carcinomas and ovarian carcinomas and in some colon and kidney carcinomas. P53 is also frequently overexpressed in tumors. This protein, which inhibits cell multiplication, is normally very rapidly recycled in tumor cells. Telomerase (hTERT) is found in more than 80% of tumors, irrespective of their tissue origin, whereas it is absent or expressed at low noise in normal cells. The action of the telomerase serves to compensate for the reduction in telomers which takes place during cell division. The maintaining of a constant telomer length by telomerase promotes cell proliferation and therefore tumorigenesis. Inhibitor of apoptosis proteins (IAPB), such as the survivin protein, constitute a family of proteins which, by inhibiting caspases, inhibit cell death.

Other Antigens

The other antigen categories are the antigens which result from a mutation or a genetic arrangement (MUM-1, CDK4, beta-catenin, HLA-A2, BCR-ALB, CASP-8) and the tumor antigens of viral origin (E6 and E7 proteins of papillomaviruses involved in cervical cancer).

Although many tumor antigens have already been discovered, vaccines for combating cancer are not perfected. Vaccination trials remain quite disappointing and cases of regression caused by vaccines are rare. These failures are the result of a weak immunogenicity of the antigens identified or of escape mechanisms which mean that the tumor no longer expresses the target antigen. The antigens targeted are often not vital for the cell, so that tumor escape can occur. The antigens which have been identified have been done so mainly on melanomas and are not suitable for the numerous other cancers. There are few known antigens which have a broad spectrum of expression and which make it possible to have a vaccine suitable for many cancers. These are mainly the overexpressed antigens such as telomerase and survivin (PCT international application WO 2007/036638).

However, there are many proteins that are preferentially expressed in tumor cells, irrespective of their origin, and which could therefore constitute vaccines suitable for many cancers. In order for these proteins to be of interest for vaccines, it is necessary to show that they induce T lymphocytes capable of recognizing tumor cells which express these proteins. It is in fact possible that they are only weakly immunogenic owing to mechanisms of tolerance or the absence of T epitopes in their sequence. It is also possible that they are capable of inducing an immune response, but that the cells induced do not recognize the tumors. The T epitopes derived from these proteins may in fact not be presented at the surface of the tumor cells owing to an insufficient level of expression or incorrect processing of the proteins in the tumor cells.

The midkine (MDK) protein, also known as NEGF2 (Neurite outgrowth-promoting factor 2), was demonstrated in 1988, as an embryonic carcinoma cell protein induced by retinoic acid (Kadomatsu et al., Biochem. Biophys. Res. Commun. 1988, 151, 1312-1318; for a review, see http://www.midkine.org). In humans, the midkine gene is located on chromosome 11 at position 11p11.2. It comprises 4 exons and has a size of 3.5 kb; the coding sequence corresponds to NCBI accession number M69148 (SEQ ID NO: 1 in the appended sequence listing). The regulatory 5′ region contains a retinoic acid response site and two WT1 (Wilms Tumor Supression 1) tumor suppressor response sites. The retinoic acid response site is responsible for the induction of midkine expression by retinoic acid, while the WT1 response sites are involved in the decreasing of expression by WT1. A human midkine protein splice variant, known as INSP106, has also been described (PCT international application WO 2004/052928).

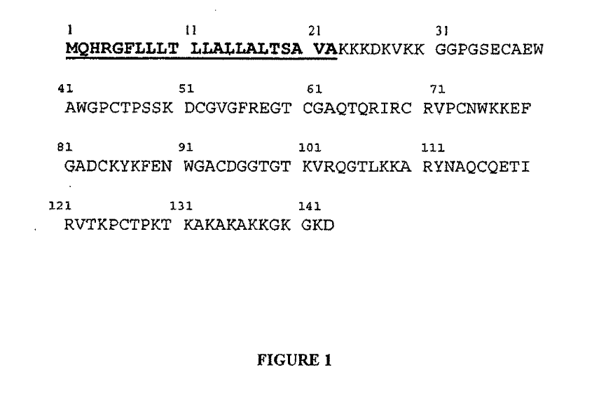

Midkine is a 143 amino acid protein rich in basic residues which has five disulfide bridges [(37,61); (45,70); (52,74); (84,116); (94,126)]. The human sequence corresponds to SwissProt accession number P21741 (FIG. 1 and SEQ ID NO: 2 in the appended sequence listing). It is expressed in the form of a precursor comprising a signal peptide and 22 amino acids (FIG. 1). It exhibits approximately 50% homology with the pleiotrophin protein. The structure of midkine was resolved by NMR in 1997. The protein comprises two different domains, each made up of three anti-parallel beta-sheets maintained by disulfide bridges; the two domains are connected by a flexible region. The biological activity (neurite growth, fibrinolysis and nerve cell migration) requires only the C-terminal domain. This domain is conserved and is found from drosophila to humans, which confirms its functional role. It also comprises two heparin-binding sites. At least four receptors capable of binding midkine are known, which gives it many activities: the members of the syndecan family, which are proteoglycans comprising heparin sulfates; PTP, which is a proteoglycan comprising chondroitin sulfate; ALK (Anaplastic Lymphoma Kinase); LRP, which is a member of the LDL receptor family.

In a normal individual, midkine is mainly expressed during embryogenesis, with an expression peak in the middle of gestation. Midkine is involved in neuron development. It causes neurite growth and nerve cell migration. It is also involved in the development of the neuromuscular junction and the protection of neurons. During embryogenesis, midkine is involved in the development of the teeth, lungs, kidneys and bone. Mice deficient for the midkine gene are viable and they are affected only in terms of neuronal functions, in accordance with the role of midkine in nervous system development. It has also been observed that mice made deficient for the midkine gene are less affected than control mice by nephrite induction. They are also less subject to restenosis (narrowing of the arteries due to proliferation of damaged arterial tissues).

Midkine is overexpressed in many tumors, whereas in healthy adult individuals, it is expressed less and locally (small intestine, brain). Midkine is one of the 40 genes most expressed in tumors compared with healthy tissues (Velculescu et al., Nat. Genet., 1999, 23, 387-388). Midkine is overexpressed in approximately 80% of cases of numerous human cancers, in particular carcinomas. High expression of midkine has been observed in particular in esophageal, stomach, colon, pancreatic, thyroid, lung, breast, bladder, uterine, ovarian and prostate cancers, hepatocellular carcinomas, osteosarcomas, neuroblastomas, glioblastomas, astrocytomas, leukemias and Wilms tumors (Moon et al., Gynecologic Oncology, 2003, 88, 289-297; Hidaka et al., Leukemia Res., 2007, 8, 1045-1051; Maeda et al., Br. J. Cancer, 2007, 97, 405-411; Ren et al., World J. Gastroenterol., 2006, 12, 2006-2010). A high expression has been correlated with poor prognosis in bladder cancers, glioblastomas and neuroblastomas (O'Brien, Cancer Res., 1996, 56, 2515-2518). In addition, the overexpression of midkine is correlated with an increased resistance to chemotherapy in human gastric cancer cell lines. Midkine is not only expressed in tissues. A high level of midkine has been observed in the serum of more than 60% of patients suffering from carcinomas (Muramatsu et al., J. Biochem., 2003, 132, 259-371). This level decreases when the tumor is removed. The presence of midkine in the serum could therefore have a diagnostic value. Midkine appears to have many activities in relation to tumorigenesis. It in fact has a transforming, anti-apoptotic, mitogenic, angiogenic, fibrinolytic and chemotactic activity (Kadomatsu et al., Cancer Letters, 2004, 127-143). It has been shown that an antisense strategy targeting the midkine gene suppresses tumorigenesis of a carcinoma in mice (Takei et al., Cancer Research, 2001, 61, 8486-8491).

Owing to its many biological activities, midkine or modulators (inhibitors) thereof is (are) of use for stimulating angiogenesis and hematopoiesis, preventing atherosclerosis and restenosis, and inhibiting apoptosis, and in the prevention and treatment of inflammatory, cardiac (myocardial infarction), cerebral, hepatic, nerve, renal, ocular (retinopathies), neurofibromatous, respiratory (asthma and pulmonary hyperplasia) and post-surgical pathological conditions (United States applications US 2003/0072739, US 2003/0185794, US 2004/0077579, US 2005/0079151, US 2006/0148738 and US 2005/0130928; European patent application EP 1832296, PCT international applications WO 2007/055397 and WO 2000/031541; and U.S. Pat. No. 5,629,284; U.S. Pat. No. 6,383,480 and U.S. Pat. No. 6,572,851).

In addition, owing to the frequent expression of midkine in tumors, combined with the presence of the protein in the blood and urine, and also the existence of a midkine polymorphism associated with the risk of cancer, midkine represents a marker for evaluation of the risk and the diagnosis and prognosis of cancer (U.S. Pat. No. 7,090,983 and applications US 2003/0149534 and US 2004/0219614). Midkine is in particular detected using monoclonal antibodies specific for a truncated midkine corresponding to positions 23 to 25 and 82 to 143 of the midkine precursor (United States application US 2004/0219614). The midkine promoter is also used in suicide gene strategies.

On the other hand, the immunogenicity of the midkine protein has not been studied.

The inventors have shown that the midkine protein, which has a preferential expression in tumors, contains peptides capable of inducing specific CD4⁺ T and/or CD8⁺ T lymphocytes that recognize the midkine protein expressed by tumor cells in many types of cancers, in the majority of individuals of the caucasian population. These peptides represent potential candidates for prophylactic or therapeutic vaccination against cancers, given that they are capable of inducing a CD4⁺ T and CD8⁺ T response directed against the tumor, in the majority of vaccinated patients, since: (i) they are derived from an antigen expressed by many tumors, (ii) they are capable of inducing specific CD4⁺ T and CD8⁺ T lymphocytes that recognize the antigen expressed by the tumors, and (iii) they are recognized by CD4⁺ T and CD8⁺ T lymphocytes in the majority of individuals of the caucasian population owing to the fact that they take into account the polymorphism of the HLA molecules and are restricted by the HLA molecules predominant in the caucasian population.

In addition, these peptides, which are recognized by CD4⁺ T and/or CD8⁺ T lymphocytes specific for a tumor antigen expressed by the majority of tumors, are of use for immunomonitoring of the cellular response against midkine over the course of the progression of a cancer and in particular after an anticancer treatment (surgical, chemotherapy, radiotherapy, immunotherapy).

Consequently, the subject of the present invention is the use of a peptide derived from the midkine protein, comprising at least one CD4⁺ T or CD8⁺ T epitope restricted by the HLA molecules predominant in the caucasian population, or of a polynucleotide encoding said peptide, for the preparation of an anticancer vaccine, intended for the treatment of cancers associated with tumor overexpression of said midkine protein.

### DEFINITIONS

The term “peptide derived from midkine” is intended to mean both the midkine protein (precursor of 143 amino acids or mature protein (positions 23 to 143 of the precursor)) and a peptide fragment of at least 8 consecutive amino acids of said protein. The term “midkine” is intended to mean a midkine protein derived from any mammal; it is preferably the human protein. The positions of the peptides derived from midkine are indicated with reference to the human sequence (SwissProt P21741, FIG. 1 and SEQ ID NO: 2).
The term “HLA molecule predominant in the caucasian population” or “predominant HLA molecule” is intended to mean a predominant HLA I (HLA-A, HLA-B or HLA-C) or HLA II molecule. It involves the HLA-A, HLA-B and HLC-C molecules comprising an alpha chain encoded by an allele of which the frequency is greater than 5% in the caucasian population, as specified in table I below.

TABLE I
Gene (allele*)/phenotype frequency of HLA I

| | Europe | | USA | | Africa | Asia | |
|---|---|---|---|---|---|---|---|
| Alleles | France | Germany | Caucasian | Afro-american | Senegal | India | Japan |
| A1 | 14.6/27.1 | 17/31.1 | 16.6/30.4 | 5.3/10.3 | 4.9/9.6 | 11.1/21.0 | 0.7/1.4 |
| A2 | 20.9/37.4 | 26.6/46.1 | 27.9/48.0 | 17.3/31.6 | 18.6/33.7 | 12.1/22.7 | 24.1/42.4 |
| A3 | 9.2/17.6 | 14.2/26.4 | 11.4/21.5 | 8.9/17.0 | 5.8/11.3 | 7.9/15.2 | 0.6/1.2 |
| A11 | 5.7/11.1 | 5.5/10.7 | 5.3/10.3 | 2.6/5.1 | | 15.9/29.3 | 10.4/19.7 |
| B7 | 7.4/14.3 | 11.1/21.0 | 9.8/18.6 | 8/15.4 | 4.4/8.6 | 9.5/18.1 | 5/9.8 |
| B8 | 7.6/14.6 | 9.4/17.9 | 10/19.0 | 3.1/6.1 | 6/11.6 | 3.8/7.5 | |
| B18 | 5.2/10.1 | 3.7/7.3 | 4.7/9.2 | 3.2/6.3 | 4.5/8.8 | 2.5/4.9 | |
| B27 | 3.4/6.7 | 3.9/7.6 | 3.9/7.6 | 1.8/3.6 | 1.9/3.8 | 2.8/5.5 | 0.4/0.8 |
| B35 | 8.2/15.7 | 9/17.2 | 8.6/16.5 | 8.3/15.9 | 13.9/25.9 | 12/22.6 | 8.1/15.5 |
| C2 | 5.1/9.9 | 7.7/14.8 | 5.4/10.5 | 10.1/19.2 | 7.6/14.6 | 2.5/4.9 | 12.2/22.9 |
| C4 | 10.9/20.6 | 11.8/22.2 | 9.6/18.3 | 21.2/37.9 | 18.1/32.9 | 14/26.0 | 4.3/8.4 |
| C7 | 20.9/37.4 | 28.6/49.0 | 21.6/38.5 | 18.2/33.1 | 12.5/23.4 | 11.2/21.1 | 1.1/2.2 |

*The predominant HLA I molecules (gene frequency > 5%) are indicated in bold

It also involves the HLA II molecules comprising a beta chain encoded by an allele of which the frequency is greater than 5% in the caucasian population, as specified in table II below.

TABLE II
Gene (allele*)/phenotype frequency of HLA II

| | Europe | | USA | | Africa | Asia | |
|---|---|---|---|---|---|---|---|
| Alleles | France | Germany | Caucasian | Afro-american | Senegal | India | Japan |
| DRB1*0101 | 9.3/17.7 | 6.7/13 | 7.3/14.1 | 1.9/3.8 | 0.6/1.2 | 3.8/7.5 | 4.9/9.6 |
| DRB1*0401 | 5.6/10.9 | 8.1/15.5 | 6.7/13 | 1.5/3.0 | 0/0 | 0.9/1.8 | 0/0 |
| DRB1*1101 | 9.2/17.6 | 9.2/17.6 | 4.4/8.6 | 8.2/15.7 | 9.3/17.7 | 0.9/1.8 | 2/4 |
| DRB1*0701 | 14.0/26 | 12.3/23.1 | 14.4/26.7 | 9.8/18.6 | 7.8/15 | 13/24.3 | 0.6/1.2 |
| DRB1*0301 | 10.9/20.6 | 9.4/17.9 | 9.5/18.1 | 7/13.5 | 10.2/19.4 | 5.3/10.3 | 0.4/0.8 |
| DRB1*1301 | 6.0/11.6 | 4.5/8.8 | 5.1/9.9 | 4.2/8.2 | 4.7/9.2 | 6.3/12.2 | 0.7/1.4 |
| DRB1*1501 | 8.0/14.4 | 7.8/15 | 10.3/19.5 | 8.6/16.5 | 0/0 | 12.1/22.7 | 9.1/17.4 |
| TOTAL | 63.0/86.3 | 58.0/82.4 | 57.7/82.1 | 41.2/65.4 | 32./54.66 | 42.3/66.7 | 17.7/32.3 |
| DRB5*0101 | 7.9/15.2 | 4.6/9 | 2.4/4.7 | 10.4/19.7 | 0/0 | 0/0 | 5.6/10.9 |
| DRB3*0101 | 9.2/17.6 | 9.8/18.6 | 10.4/19.7 | 15.1/27.9 | 6.9/13.3 | 4.9/9.6 | 6.5/12.6 |
| DRB4*0101 | 28.0/48.2 | 21.1/37.7 | 19.8/35.7 | 16.5/30.3 | 6.9/13.3 | 24.8/43.4 | 28.9/49.4 |
| TOTAL | 45.1/69.9 | 35.5/58.4 | 32.6/54.6 | 42.0/66.4 | 13.8/25.7 | 29.7/50.6 | 41.0/65.2 |
| DPB1*0101 | 7.1/13.7 | 2.2/4.4 | 3.2/6.3 | 27.7/47.7 | 18.2/33.1 | | 0.1/0.2 |
| DPB1*0201 | 11.9//22.4 | 8.5/16.3 | 9.8/18.6 | 12.9/24.1 | 13.8/25.7 | | 20.6/37 |
| DPB1*0301 | 17.0/31.1 | 3.8/7.5 | 7.4/14.3 | 3.3/6.5 | 3.8/7.5 | | 3/5.9 |
| DPB1*0401 | 40.0/64 | 38.1/61.7 | 25.1/43.9 | 11/20.8 | 4.8/9.4 | | 4.7/9.2 |
| DPB1*0402 | 11.0/20.8 | 15.4/28.4 | 12.6/23.6 | 9/17.2 | 25.5/44.5 | | 36.8/60.1 |
| TOTAL | 87.0/98.3 | 68.0/89.8 | 58.1/82.4 | 63.9/87.0 | 66.1/88.5 | | 65.2/87.9 |
| DP401 + 402 | 51/76 | 53.5/78.4 | 37.7/61.2 | 20/36.0 | 30.3/51.4 | | 41.5/65.8 |

*The predominant HLA II molecules (gene frequency > 5%) are indicated in bold

Some of the HLA molecules predominant in the caucasian population, and in particular the HLA-DP401 and HLA-DP402 molecules, are also predominant in other populations (South America, India, Japan, Africa; table II). Consequently, the peptides according to the invention are not restricted to use in the caucasian population, and they can also be used for vaccinating individuals from countries other than those of North America and Europe, in which said HLA molecules are predominant, as specified in table II.
For the purpose of the present invention, the terms “prevailing”, and “predominant” are considered to be equivalent and are used without distinction.
The expression “CD4⁺ T epitope of midkine restricted by HLA II molecules predominant in the caucasian population” is intended to mean a peptide of 11 to 15 amino acids which binds at least one HLA II molecule predominant in the caucasian population and which is recognized by CD4⁺ T lymphocytes in the individuals of this population; the peptide comprises a sequence of 9 amino acids including the residues for anchorage to the HLA II molecules, flanked at one of its ends, preferably at both ends, by at least two amino acids, preferably 3 amino acids.
The expression “CD8⁺ T epitope midkine restricted by HLA I molecules predominant in the caucasian population” is intended to mean a peptide of 8 to 13 amino acids which binds at least one HLA I molecule predominant in the caucasian population and which is recognized by CD8⁺ T lymphocytes in the individuals of this population; the peptide comprises a sequence of 8 or 9 amino acids including the residues for anchorage to the HLA I molecules.
The term “cancer” is intended to mean a cancer associated with overexpression of the midkine protein by tumor cells, such as, in a nonlimiting manner: esophageal, stomach, colon, pancreatic, thyroid, lung, breast, bladder, uterine, ovarian and prostate cancers, heptacellular carcinomas, osteosarcomas, neuroblastomas, glioblastomas, astrocytomas, leukemias and Wilms tumors.
The term “natural or synthetic amino acid” is intended to mean the 20 natural α-amino acids commonly found in proteins (A, R, N, D, C, Q, E, G, H, I, L, K, M, F, P, S, T, W, Y and V), some amino acids rarely encountered in proteins (hydroxyproline, hydroxylysine, methyllysine, dimethyllysine, etc.), amino acids which do not exist in proteins, such as β-alanine, γ-aminobutyric acid, homocysteine, ornithine, citrulline, canavanine, norleucine, cyclohexylalanine, etc., D amino acids derived from the L amino acids, and the analogs of the above amino acids.
The term “hydrophobic amino acid” is intended to mean an amino acid selected from (one-letter code): A, V, L, I, P, W, F and M.
The term “aromatic amino acid” is intended to mean an amino acid selected from (one-letter code): F, W and Y.

The peptides according to the invention are recognized by CD4⁺ T and/or CD8⁺ T lymophocytes in the majority of individuals since they are presented by HLA I and HLA II molecules which are predominant in the caucasian population. They are immunogenic, i.e. they are capable of inducing midkine-specific CD4⁺ T and/or CD8⁺ T lymphocytes from the precursors present in the majority of naïve individuals or else of stimulating such T lymphocytes in the majority of individuals who have a cancer associated with the overexpression of midkine. In addition, the CD4⁺ T and/or CD8⁺ T lymphocytes which are induced in the majority of individuals recognize the midkine expressed by the tumors of these individuals. The immunogenicity of the peptides can be determined, in particular using peripheral blood mononuclear cells (PBMCs), by any suitable assay known to those skilled in the art, for instance: a cell proliferation test, a cytotoxicity test, an Elispot test (assaying of cytokine-producing cells) or a test for assaying cytokines (IFN-γ, IL-2, IL-4, IL-10, IL-5, TNF-α and TGF-β.

The invention encompasses the natural or synthetic variant peptides obtained by mutation (insertion, deletion, substitution) of one or more amino acids in the midkine sequence, provided said sequence conserves good affinity for the predominant HLA molecules and is immunogenic. The natural variants result in particular from the polymorphism of midkine. In addition, other variants can be readily constructed, given that the amino acid residues involved in binding to the HLA-DR and HLA-DP4 molecules (anchoring residues) and the effect of modifications of these residues on binding to the HLA-DR and HLA-DP4 molecules are known to those skilled in the art; PCT international application WO 03/040299 teaches in particular that, in order to bind HLA-DP4, the residue at P6 should be aromatic or hydrophobic or consist of a cysteine residue (C), and at least one of the residues P1 and P9 is such that P1 is aromatic or hydrophobic and/or P9 is aromatic or hydrophobic or consists of a C, D, Q, S, T or E residue, whereas the residue at P4 can be any amino acid residue. U.S. Pat. No. 6,649,166 describes a general method for determining the residues for anchorage to the HLA-DR molecules (P1, P4, P6, P7 and P9) and the nature of the mutations of these residues which make it possible to modify the affinity for the HLA-DR molecules. HLA-DR molecule-binding motifs are described in particular in Sturnolio et al., Nat. Biotech., 1999, 17, 533-534 and Rammensee et al., Immunogenetics, 1995, 41, 178-228.

The amino acid residues involved in binding to the HLA-I molecules (anchoring residues) and the effect of the modifications of these residues on binding to the HLA-I molecules are known to those skilled in the art. The motifs for binding of the peptides to the class I HLA molecules are described in Rammensee et al., Immunogenetics, 1995, 41, 178-228 and in table III below.

TABLE III
Motifs for binding of the main HLA-A* alleles

| | positions | | | | | | | | |
|---|---|---|---|---|---|---|---|---|---|
| Alleles | 1 | 2 | 3 | 4 | 5 | 6 | 7 | 8 | 9 |
| A1 | | T, S | D, E | | | | L | | Y |
| A2 | | L, M | | | | V | | | V, L |
| A3 | | L, V, | F, Y | | | I, M, F, | I, M, L, | | K, Y, |
| | | M | | | | V, L | F | | F |
| A11 | | V, I, | M, L, F, | | | | L, I, Y, | | K, R |
| | | F, Y | Y, I | | | | F, V | | |

*The major anchoring residues are in bold.

It is also known that certain substitutions improve the affinity of peptides for the HLA I molecules without disturbing their antigenicity; this is the case of the introduction of a tyrosine at position 1 on an HLA-A2-binding peptide (Tourdot et al., Eur. J. Immunol., 2000, 30, 3411-3421).

The invention also encompasses the modified peptides derived from the peptides above by introduction of any modification at the level of amino acid residue(s), of the peptide binding or of the ends of the peptides, provided that said modified peptide conserves good affinity for the predominant HLA molecules and is immunogenic. These modifications which are introduced into the peptides by conventional methods known to those skilled in the art include, in a non-limiting manner: the substitution of an amino acid with a non-proteinogenic amino acid (D amino acid or amino acid analog); the addition of a chemical group (lipid, oligosaccharide or polysaccharide) at the level of a reactive function, in particular of the side chain R; the modification of the peptide bond (—CO—NH—), in particular with a bond of the retro or retro-inverso type (—NH—CO—) or a bond other than the peptide bond; cyclization; fusion of a peptide (epitope of interest for vaccination; tag of use for purification of the peptide, in particular in a form cleavable by a protease); fusion of the sequence of said peptide with that of a protein, in particular an α-chain of an HLA I or HLA II molecule, a β-chain of an HLA II molecule or the extracellular domain of said chain or alternatively a sequence for targeting to the endosome, derived in particular from the invariable chain Ii or from the LAMP-1 protein; coupling to a suitable molecule, in particular a label, for example a fluorochrome or biotin. These modifications are intended in particular to increase the stability and more particularly the resistance to proteolysis, and also the solubility or the immunogenicity or to facilitate the purification or the detection either of the peptide according to the invention or of CD4⁺ and/or CD8⁺ cells specific for said peptide.

According to one advantageous embodiment of said use, said peptide consists of the midkine protein. Preferably, it is the human protein of sequence SEQ ID NO: 2.

The present invention encompasses the use of the midkine protein denatured by any suitable means known to those skilled in the art, and in particular the reduced midkine protein.

The present invention also encompasses the use of variants of the midkine protein, in which at least one of the cysteines involved in a disulfide bridge is replaced with another amino acid, for example a serine.

The present invention also encompasses the use of peptides of at least 8 amino acids derived from the midkine protein, which comprise at least one CD4⁺ T or CD8⁺ T epitope as defined above. The invention encompasses the use of peptides which bind one of the HLA I molecules and/or one of the HLA II molecules most frequent in the caucasian population, in particular the HLA-A2 molecule (table I) and/or the HLA-DR7, HLA-DRB4, HLA-DP401 or HLA-DP402 molecules (table II). The invention also encompasses the use of peptides which bind several different predominant HLA I and/or HLA II molecules, so as to broaden the vaccine coverage to the majority of the caucasian population.

The invention also encompasses the use of peptides of at least 8 amino acids of the N-terminal domain of midkine (positions 1 to 84 with reference to the midkine precursor sequence) which comprise at least one CD4⁺ T or CD8⁺ T epitope as defined above.

In accordance with the invention, said fragment has a length of from 8 to 100 amino acids, preferably from 8 to 50 amino acids, preferably from 10 to 25 amino acids (10, 11, 12, 13, 14, 15, 16, 17, 18, 19, 20, 21, 22, 23, 24 or 25 amino acids).

According to another advantageous embodiment of said use, said peptide is a fragment of at least 8 amino acids of the midkine protein, comprising at least one HLA-A2 molecule-restricted CD8⁺ T epitope, said peptide comprising at least positions 14 to 21 or 114 to 122 of the amino acid sequence of said midkine protein.

Preferably, said peptide comprises positions 12 to 21, 13 to 21, 13 to 22, 14 to 22 or 113 to 122 of the amino acid sequence of said midkine protein.

Preferably, said peptide consists of positions 12 to 21 (MDK 12-21), 13 to 21 (MDK 13-21), 13 to 22 (MDK 13-22), 14 to 22 (MDK 14-22), 113 to 122 (MDK 113-122) or 114 to 122 (MDK 114-122) of the amino acid sequence of the midkine protein; these peptides correspond, respectively, to the sequences SEQ ID NO: 3 to 8 in the appended sequence listing.

According to another advantageous embodiment of said use, said peptide is a fragment of at least 8 amino acids of the midkine protein, comprising at least one CD4⁺ T epitope restricted by at least one HLA II molecule predominant in the caucasian population, said peptide comprising at least positions 9 to 15, 14 to 28, 52 to 64, 64 to 78, 70 to 84, 74 to 88, 78 to 92, 84 to 98, 99 to 113, 105 to 119, 110 to 124 or 119 to 133 of the amino acid sequence of said midkine protein. Examples of these peptides are the peptides of sequence SEQ ID NO: 9, 10, 13 to 15, 21 to 26, 28, 29 and 30 (table VII).

Preferably, said HLA II molecule predominant in the caucasian population is chosen from the HLA-DR1, HLA-DR3, HLA-DR4, HLA-DR7, HLA-DR11, HLA-DR13, HLA-DR15, HLA-DRB3, HLA-DRB4, HLA-DRB5, HLA-DP40 and HLA-DP402 molecules. Said HLA II molecules are advantageously encoded, respectively, by the HLA DRB1*0101, DRB1*0301, DRB1*0401, DRB1*0701, DRB1*1101, DRB1*1301, DRB1*1501, DRB3*0101, DRB4*0104, DRB5*0101, DP*0401 and DP*0402 alleles.

Preferably, said peptide binds at least four different HLA II molecules predominant in the caucasian population and comprises at least positions 9 to 15, 14 to 28 or 110 to 124 of the amino acid sequence of said midkine protein. Said peptide advantageously comprises positions 1 to 15, 4 to 18, 9 to 21, 9 to 22, 9 to 23 or 14 to 28 of the amino acid sequence of the midkine protein.

Preferably, said peptide consists of positions 1 to 15 (MDK 1-15), 4 to 18 (MDK 4-18), 9 to 21 (MDK 9-21), 9 to 22 (MDK 9-22), 9 to 23 (MDK 9-23), 14 to 28 (MDK 14-28) or 110 to 124 (MDK 110-124) of the amino acid sequence of the midkine protein; these peptides correspond, respectively, to the sequences SEQ ID NO: 9 to 15 in the appended sequence listing.

In accordance with the invention, said peptide advantageously comprises several CD4⁺ and/or CD8⁺ T epitopes of the midkine protein, optionally combined with other CD4⁺ T, CD8⁺ T or B epitopes. The epitopes are advantageously CD4⁺ T or CD8⁺ T epitopes derived from tumor antigens as described on the site http:www/cancerimmunity.org/peptidedatabase/tumorspecific.htm in particular CD4⁺ T or CD8⁺ T epitopes derived from MAGE, NY-ESO-1 or survivin.

According to one advantageous arrangement of the above embodiments, said peptide is a fragment of the midkine protein, comprising at least one CD8⁺ T epitope restricted by the HLA-A2 molecule and at least one CD4⁺ T epitope restricted by at least four different HLA II molecules predominant in the caucasian population, said peptide comprising positions 9 to 21, 9 to 22, 9 to 23 or 110 to 124 of the amino acid sequence of said midkine protein. Preferably, said peptide consists of positions 9 to 21 (MDK 9-21), 9 to 22 (MDK 9-22), 9 to 23 (MDK 9-23) or 110 to 124 (MDK 110-124) of the amino acid sequence of the midkine protein.

Such a peptide advantageously makes it possible to induce both CD4⁺ T lymophocytes and CD8⁺ T lymphocytes specific for many tumors in the majority of individuals of the caucasian population who have these tumors.

The various epitopes can be included in the vaccine composition in the form of a mixture of isolated peptides, of a multi-epitope peptide, of a fusion protein or of a polynucleotide encoding the above peptides/protein. Said peptides/protein can be modified or associated with liposomes or lipids, in particular in the form of lipopeptides. Preferably, said polynucleotide is included in a vector, in particular an expression vector.

Among the epitopes which can be incorporated into the vaccine composition of the invention, mention may in particular be made of:
the CD8⁺ T epitopes of MAGE, as described in U.S. Pat. No. 6,063,900 and PCT application WO 2004/052917,
the CD4⁺ T epitopes of MAGE, such as DR1-restricted MAGE-A3 267-282 (PCT international application WO 02/095051); DR4- and DR7-restricted MAGE-A3 149-160 (Kobayashi et al., Cancer Research, 2001, 61, 4773-4788); DR11-restricted MAGE-A3 191-205 and 281-295 (Consogno et al., Blood, 2003, 101, 1038-1044; Manici et al., J. Exp. Med., 1999, 189, 871-876) and DR13-restricted MAGE-A3 121-134 (U.S. Pat. No. 6,716,809); DR15-restricted MAGE-A1 281-292 (PCT international application WO 00/78806); DR4-restricted MAGE-A6 102-116, 121-144, 140-170, 145-160, 150-165 and 246-263 (Tatsumi et al., Clinical Cancer Research, 2003, 9, 947-954); DR15-restricted MAGE-A1 281-292 (PCT international application WO 00/78806); DR4-restricted MAGE-A6 102-116, 121-144, 140-170, 145-160, 150-165 and 246-263 (Tatsumi et al., Clinical Cancer Research, 2003, 9, 947-954) and the HLA-DP4-restricted MAGE epitopes as described in PCT international application WO 2007/026078,
a CD8⁺ T epitope of survivin, chosen from: survivin 96-104 (LTLGEFLKL, SEQ ID NO: 39) or 95-104 (ELTLGEFLKL, SEQ ID NO: 40), survivin-2B 80-88 (AYACNTSTL, SEQ ID NO: 41) and the peptides as described in table I of Bachinsky et al., Cancer Immun., 2005, 5, 6-,
A CD4⁺ T epitope of survivin as described in PCT international application WO 2007/036638, and in particular peptide 19-33, 90-104 or 93-107,
a natural or synthetic universal CD4⁺ T epitope, such as the tetanus toxin peptide TT 830-846 (O'Sullivan et al., J. Immunol., 1991, 147, 2663-2669), the flu virus hemagglutinin peptide HA 307-319 (O'Sullivan et al., mentioned above), the PADRE peptide (KXVAAWTLKAA, SEQ ID NO: 16; Alexander et al., Immunity, 1994, 1, 751-761) and peptides derived from the antigens of Plasmodium falciparum, such as the CS.T3 peptide (Sinigaglia et al., Nature, 1988, 336, 778-780) and the CSP, SSP2, LSA-1 and EXP-1 peptides (Doolan et al., J. Immunol., 2000, 165, 1123-1137).
A B epitope made up of a sugar (Alexander et al., mentioned above), said B epitope preferably being in the form of a glycopeptide, and
a B epitope of midkine recognized specifically by antibodies directed against said tumor antigen.

The combination of midkine CD4⁺ T and/or CD8⁺ T epitope(s) with at least one of the epitopes as defined above advantageously makes it possible to improve the antitumor immune response, and in particular to establish a long-term immune memory.

According to another advantageous embodiment of said use, said peptide derived from midkine is a multi-epitope peptide comprising the concatenation of at least two identical or different epitopes, at least one of which is a midkine CD4⁺ T and/or CD8⁺ T epitope. The multi-epitope peptide advantageously comprises other epitopes (CD4⁺ T or CD8⁺ T epitope of another tumor antigen), as defined above. In accordance with the invention, the sequences of the various epitopes are linked to one another by a peptide bond or separated by heterologous sequences, i.e. sequences different than those naturally present at this position in the amino acid sequence of midkine. Preferably, said multi-epitope peptide has a length of from 20 to 1000 amino acids, preferably from 20 to 100 amino acids.

Said multi-epitope peptide advantageously comprises a tag fused to one of its ends, for the purification or the detection of said fragment. The tag, in particular a polyhistidine sequence or a B epitope of an antigen, is preferably separated from the multi-epitope sequence by a cleavage site for a protease so as to isolate the multi-epitope sequence, from the fusion.

According to another advantageous embodiment of said use, said peptide derived from midkine is a lipopeptide comprising a multi-epitope fragment or peptide, as defined above.

Said lipopeptide is in particular obtained by addition of a lipid to an α-amino function or to a reactive function of the side chain of an amino acid of said multi-epitope fragment or peptide; it may comprise one or more chains derived from C₄₋₂₀ fatty acids, which are optionally branched or unsaturated (palmitic acid, oleic acid, linoleic acid, linolenic acid, 2-aminohexadecanoic acid, pimelautide, trimexautide) or a derivative of a steroid. The preferred lipid portion is in particular represented by an N^{α}-acetyllysin N^{ε} (palmitoyl) group, also called Ac-K(Pam).

According to another advantageous embodiment of said use, said peptide derived from midkine is fused with a heterologous protein or protein fragment (fusion protein).

The multi-epitope fragment or peptide can be fused with the NH₂ or COOH end of said protein, or inserted into the sequence of said protein. According to one advantageous embodiment of said fusion protein, it consists of a peptide as defined above, fused with a sequence for targeting to the endosome, preferably derived from a human invariable chain Ii or from the LAMP-1 protein. The sequences for targeting to the endosome and their use for targeting antigens to the endosome are in particular described in Sanderson et al. (Proc. Nat. Acad. Sci., USA, 1995, 92, 7217-7222), Wu et al. (Proc. Nat. Acad. Sci., USA, 1995, 92, 11671-11675) and Thompson et al. (J. Virol., 1998, 72, 2246-2252).

According to an advantageous arrangement of said fusion protein, it consists of a peptide as defined above, fused with one of the chains of an HLA molecule, preferably the beta-chain of an HLA II molecule or the alpha-chain of an HLA I molecule, or else with a fragment thereof corresponding to a soluble HLA molecule, in particular a fragment corresponding to the extracellular domain preceded by the homologous signal peptide or by a heterologous signal peptide. Said peptide is advantageously inserted between the signal peptide and the NH₂ end of the extracellular domain of the α- or β-chain, as described for the HLA-DR molecule (Kolzin et al., PNAS, 2000, 97, 291-296).

Alternatively, said multi-epitope fragment or peptide is fused with a protein which facilitates its purification or its detection, known to those skilled in the art, such as in particular glutathione-S-transferase (GST) and the fluorescent proteins (GFP and derivatives). In this case, the sequence of the multi-epitope fragment or peptide of interest is preferably separated from the rest of the protein by a cleavage site for a protease, in order to facilitate the purification of said multi-epitope fragment or peptide.

According to another advantageous embodiment of said use, said polynucleotide encodes a peptide, a multi-epitope fragment or a fusion protein, as defined above.

In accordance with the invention, the sequence of said polynucleotide is that of the cDNA encoding said multi-epitope fragment or peptide or said fusion protein. Said sequence can advantageously be modified in such a way that the codon usage is optimal in the host in which it is expressed. In addition, said polynucleotide can be linked to at least one heterologous sequence.

For the purpose of the present invention, the expression “heterologous sequence relative to a nucleic acid sequence encoding midkine” is intended to mean any nucleic acid sequence other than those which, naturally, are immediately adjacent to said nucleic acid sequence encoding said midkine peptide.

Preferably, said polynucleotide is inserted into a vector.

For the purpose of the present invention, the term “vector” is intended to mean a nucleic acid molecule capable of transporting another nucleic acid with which it is combined. One type of vector which can be used in the present invention includes, in a nonlimiting manner, a linear or circular DNA or RNA molecule consisting of chromosomal, nonchromosomal synthetic or semi-synthetic nucleic acids, such as, in particular, a viral vector, a plasmid vector or an RNA vector.

Many vectors into which a nucleic acid molecule of interest can be inserted in order to introduce it into and maintain it in a eukaryotic or prokaryotic host cell are known in themselves: the choice of a suitable vector depends on the use envisioned for this vector (for example replication of the sequence of interest, expression of this sequence, maintaining of this sequence in extrachromosomal form, or else integration into the chromosomal material of the host), and also on the nature of the host cell. For example, it is possible to use naked nucleic acids (DNA or RNA) or viral vectors such as adenoviruses, retroviruses, lentiviruses and AAVs into which the sequence of interest has been inserted beforehand; it is also possible to combine said sequence (isolated or inserted in a plasmid vector) with a substance which allows it to cross the membrane of the host cells, such as a transporter, for instance a nanotransporter or a preparation of liposomes, or of cationic polymers, or else to introduce it into said host cell using physical methods such as electroporation or microinjection. In addition, these methods can advantageously be combined, for example using electroporation combined with liposomes.

Preferably, said vector comprises all the elements necessary for the expression of the peptide or of the protein as defined above. For example, said vector comprises an expression cassette including at least one polynucleotide as defined above, under the control of suitable sequences for regulating transcription and, optionally, translation (promoter, enhancer, intron, start codon (ATG), stop codon, polyadenylation signal, splice site).

The vaccine composition according to the invention advantageously comprises a pharmaceutically acceptable vehicle, a carrier substance and/or an adjuvant.

The pharmaceutically acceptable vehicles, the carrier substances and the adjuvants are those conventionally used.

The adjuvants are advantageously chosen from the group made up of: oily emulsions, mineral substances, bacterial extracts, oligonucleotides containing CpGs, saponin, alumina hydroxide, monophosphoryl lipid A and squalene.

The carrier substances are advantageously selected from the group consisting of: unilamellar or multilamellar liposomes, ISCOMs, virosomes, virus-like particles, saponin micelles, solid microspheres which are saccharide (poly(lactide-co-glycolide)) or gold-bearing in nature, and nanoparticles.

The vaccine composition comprises an effective dose of peptide/protein/lipopeptide/vector which makes it possible to obtain a prophylactic/therapeutic effect on the cancer associated with tumor overexpression of midkine, as defined above. This dose is determined and adjusted according to factors such as age, sex and weight of the individual. The vaccine composition is generally administered according to the usual vaccination protocols, at doses and for a period sufficient to induce a cellular response directed against the midkine protein. The administration may be subcutaneous, intramuscular, intravenous, intradermal, intraperitoneal, oral, sublingual, rectal, vaginal, intranasal, by inhalation or by transdermal application.

The composition is in a galenical form suitable for a chosen administration: injectable sterile solution, powder, tablets, gel capsules, suspension, syrup, suppositories, which are prepared according to the standard protocols.

According to one advantageous embodiment of said composition, it comprises at least one CD4⁺ T epitope and one CD8⁺ T epitope of midkine, in the form of a mixture of peptides, of a multi-epitope fragment and/or of an expression vector encoding said peptides or said fragment, as defined above.

According to one advantageous arrangement of this embodiment of said composition, it comprises at least the MDK 9-21, MDK 9-22, MDK 9-23 or MDK 110-124 peptide.

Preferably, the MDK 9-21, MDK 9-22 or MDK 9-23 peptide is combined with the MDK 74-88 or 78-92 peptide, with the MDK 14-28 or 99-113 peptide and with the MDK 4-18 peptide.

Such a combination of peptides which binds the HLA-A2 molecule and all of the HLA-DR1, HLA-DR3, HLA-DR4, HLA-DR7, HLA-DR11, HLA-DR13, HLA-DR15, HLA-DRB3, HLA-DRB4, HLA-DRB5, HLA-DP401 and HLA-DP402 (table VII) molecules advantageously makes it possible to induce CD4⁺ T and CD8⁺ T lymphocytes in virtually all individuals vaccinated.

According to yet another advantageous embodiment of said composition, it comprises a peptide which includes a universal CD4⁺ T epitope and/or a CD4⁺ T and/or CD8⁺ T epitope of another tumor antigen, as defined above.

The peptides according to the present invention and the derived products (multi-epitope peptide, fusion protein, lipopeptide, recombinant vector) can be used in immunotherapy in the treatment of tumors overexpressing midkine. Said peptides or derived products are used either as a vaccine, or in cell therapy, or alternatively through a combination of the two approaches.

Cell therapy comprises the preparation of antigen-presenting cells (dendritic cells) by a conventional protocol comprising the isolation of peripheral blood mononuclear cells (PBMCs) from a patient to be treated and the culturing of the dendritic cells in the presence of peptide(s). In a second step, the antigen-presenting cells loaded with the peptide are reinjected into the patient.

A subject of the present invention is also a vaccine composition, characterized in that it comprises at least one peptide fragment derived from midkine as defined above, a multi-epitope peptide, a fusion protein, a lipopeptide or a vector, as defined above, and a pharmaceutically acceptable vehicle, a carrier substance or an adjuvant.

A subject of the present invention is also a prophylactic or therapeutic antitumor vaccination method, characterized in that it comprises the administration of a vaccine composition as defined above, to an individual, by any suitable means as defined above.

A subject of the present invention is also the use of at least one peptide as defined above, for the preparation of a reagent for immunomonitoring of the cellular response against midkine, intended for evaluating the prognosis or monitoring the treatment of a cancer (surgery, radiotherapy, chemotherapy, immunotherapy). Preferably, said reagent comprises a peptide or a fusion protein as defined above, which is for example labeled and/or complexed with an HLA molecule, in the form of multimeric HLA/peptide complexes, for instance tetramers of HLA/peptide complexes, which are labeled.

A subject of the present invention is also an in vitro method for immunomonitoring of the cellular response against midkine in an individual with a cancer, characterized in that it comprises:
bringing a biological sample from said individual into contact with a peptide as defined above, and
detecting midkine-specific CD4⁺ T and/or CD8⁺ T lymphocytes by any appropriate means.

The method according to the invention makes it possible to monitor the change in the CD4⁺ T and/or CD8⁺ T response directed against midkine over the course of a cancer or else of an antitumor treatment, in particular an antitumor immunotherapy; the midkine-specific CD4⁺ T lymphocytes may be of TH1 type (secretion of IFN-γ), TH2 type (secretion of IL-4) or regulator T type (secretion of IL-10 or of TGF-β); it is expected that the TH1-type T response is the sign of a favorable progression of the cancer, whereas the regulatory T response is the sign of an unfavorable progression of this cancer. The detection is carried out using a biological sample containing CD4⁺ T and/or CD8⁺ T cells, in particular a sample of mononuclear cells isolated from a peripheral blood sample (PBMCs).

The midkine-specific CD4⁺ T and/or CD8⁺ lymphocytes are detected by any means, known in themselves. For example, use may be made of direct means such as flow cytometry in the presence of multimeric complexes as defined above, or else indirect means such as lymphocyte proliferation assays, cell cytotoxicity tests and assays for cytokines such as IL-2, IL-4, IL-5, IL-10 and IFN-γ, in particular by immunoenzymatic techniques (ELISA, RIA, ELISPOT) or by flow cytometry (assay of intracellular cytokines).

More specifically:

A suspension of cells (PBMCs, PBMCs depleted of CD4⁺ or CD8⁺ cells, T lymphocytes pre-enriched by means of an in vitro culture step with the peptides as defined above or cloned T lymphocytes) is placed in contact with said peptides and, as required, with appropriate presenting cells, such as dendritic cells, autologous or heterologous PBMCs, lymphoblastoid cells such as those obtained after infection with the EBV virus, or genetically modified cells. The presence of midkine-specific CD4⁺ T and/or CD8⁺ T cells in the initial suspension is detected by means of the peptides, according to one of the following methods:

Proliferation Assay:

The proliferation of the midkine-specific CD4⁺ T and/or CD8⁺ T cells is measured by incorporation of titrated thymidine into the DNA of the cells.

Elispot Assay:

The Elispot assay makes it possible to reveal the presence of T cells secreting cytokines (IL-2, IL-4, IL-5, IL-10, IFN-γ, TNF-α and TGF-β), specific for a peptide as defined above. The principle of this assay is described in Czerkinsky et al., J. Immunol. Methods, 1983, 65, 109-121 and Schmittel et al., J. Immunol. Methods, 1997, 210, 167-174, and its implementation is illustrated in international application WO 99/51630 or Gahéry-Ségard et al., J. Virol., 2000, 74, 1694-1703.

Detection of Cytokines:

The presence of midkine-specific T cells secreting cytokines (IL-2, IL-4, IL-5, IL-10, IFN-γ, TNF-α and TGF-β) is detected either by assaying the cytokines present in the culture supernatant, by means of an enzyme immunoassay, in particular using a commercial kit, or by detecting the intracellular cytokines by flow cytometry. The principle of detection of the intracellular cytokines is described in Goulder et al., J. Exp. Med., 2000, 192, 1819-1832 and Maecker et al., J. Immunol. Methods, 2001, 255, 27-40, and its implementation is illustrated in Draenert et al., J. Immunol. Methods, 2003, 275, 19-29.

Multimeric Complexes:
A biological sample, preferably peripheral blood mononuclear cells (PBMCs), is brought into contact with labeled multimeric complexes, in particular labeled with a fluorochrome, formed by binding between soluble HLA molecules and peptides as defined above, and
the cells labeled with said multimeric complexes are analyzed, in particular by flow cytometry.

Advantageously, prior to the biological sample being brought into contact with said complexes, it is enriched in CD4⁺ T and/or CD8⁺ T cells, by bringing it into contact with anti-CD4 or anti-CD8 antibodies.

The HLA-peptide multimeric complexes can be prepared from natural molecules extracted from cells expressing an HLA I and/or HLA II molecule or from recombinant molecules produced in appropriate host cells as specified, for example, in Novak et al. (J. Clin. Investig., 1999, 104, R63-R67) or in Kuroda et al. (J. Virol., 2000, 74, 18, 8751-8756). These HLA molecules may in particular be truncated (deletion of the transmembrane domain) and their sequence may be modified in order to make them soluble or else to facilitate the pairing of the alpha- and beta-chains (Novak et al. mentioned above).

The loading of HLA molecules with the peptide may be carried out by bringing a preparation of HLA molecules as above into contact with the peptide. For example, biotinylated soluble HLA molecules are incubated, for 72 hours at 37° C., with a 10-fold excess of peptides as defined above, in a 10 mM phosphate-citrate buffer containing 0.15 mM NaCl, at a pH of between 4.5 and 7.

Alternatively, the sequence of the peptide may be introduced into one of the chains of the HLA molecule in the form of a fusion protein which allows the preparation of HLA/peptide multimeric complexes from appropriate host cells expressing said fusion protein. Said complexes can then be labeled, in particular with biotin.

The multimeric complexes of tetramer type are in particular obtained by adding, to the loaded HLA molecules, streptavidin labeled with a fluorochrome in an amount four times less (mole for mole) with respect to the HLA molecules, the whole mixture then being incubated for a sufficient period of time, for example overnight at ambient temperature.

The multimeric complexes may also be formed either by incubation of HLA-peptide monomers with magnetic beads coupled to streptavidin, as described for HLA-I molecules (Bodinier et al., Nature, 2000, 6, 707-710), or by insertion of HLA-peptide monomers into lipid vesicles, as described for murine MHC class II molecules (Prakken, Nature Medicine, 2000, 6, 1406-1410).

To use these HLA-peptide multimeric complexes, in particular of tetramer type, a suspension of cells (PBMCs, PBMCs depleted of CD4⁺ and/or CD8⁺ cells, T lymphocytes pre-enriched by means of an in vitro culture step with peptides as defined above or cloned T lymphocytes) is brought into contact with HLA-peptide multimeric complexes at an appropriate concentration (for example, of the order of 10 to 20 μg/ml), for a period of time sufficient to allow binding between the complexes and the midkine-specific CD4⁺ and/or CD8⁺ T lymphocytes (for example, of the order of 1 to 3 hours). After washing, the suspension is analyzed by flow cytometry: the labeling of the cells is visualized by means of the multimeric complexes which are fluorescent. The flow cytometry makes it possible to separate the cells labeled with the HLA-peptide multimeric complexes from the unlabeled cells and thus to perform cell sorting.

A subject of the present invention is also an immunomonitoring reagent comprising at least one peptide as defined above. Preferably, said reagent is included in a kit. Said immunomonitoring reagent advantageously comprises a peptide or fusion protein as defined above, which is optionally labeled or complexed, in particular complexed with labeled, for example biotinylated, HLA molecules, in the form of HLA-peptide multimeric complexes, for instance tetramers of HLA-peptide complexes, which are labeled.

A subject of the present invention is thus also a method for analyzing midkine-specific CD4⁺ T and/or CD8⁺ T lymphophytes, characterized in that it comprises at least the following steps:
bringing a cell sample into contact in vitro, with labeled HLA-peptide multimeric complexes, in particular labeled with a fluorochrome, said complexes being formed by binding of soluble HLA molecules with at least one peptide as defined above, and
analyzing the cells bound to said HLA-peptide complexes, in particular by flow cytometry.

According to one advantageous embodiment of said method, the analysis of the cells (CD4⁺ T and/or CD8⁺ T lymphocytes) comprises the sorting of said cells.

A subject of the present invention is also a peptide fragment derived from midkine, a multi-epitope peptide, a fusion protein or a lipopeptide, as defined above.

A subject of the present invention is also a polynucleotide, an expression cassette, a recombinant vector, or a modified prokaryotic or eukaryotic host cell, derived from the peptides/protein above.

The invention encompasses in particular:

a) expression cassettes comprising at least one polynucleotide as defined above, under the control of appropriate regulatory sequences for transcription and, optionally, for translation (promoter, enhancer, intron, start codon (ATG), stop codon, polyadenylation signal), and

b) recombinant vectors comprising a polynucleotide in accordance with the invention. Advantageously, these vectors are expression vectors comprising at least one expression cassette as defined above.

The polynucleotides, the recombinant vectors and the transformed cells as defined above are of use in particular for the production of the peptides, multi-epitope fragments and fusion proteins according to the invention.

The polynucleotides according to the invention are obtained by the conventional methods, known in themselves, according to the standard protocols such as those described in Current Protocols in Molecular Biology (Frederick M. Ausubel, 2000, Wiley and Son Inc., Library of Congress, USA). For example, they may be obtained by amplification of a nucleic sequence by PCR or RT-PCR, by screening of genomic DNA libraries by hybridization with a homologous probe, or else by complete or partial chemical synthesis. The recombinant vectors are constructed and introduced into host cells by means of conventional recombinant DNA and genetic engineering methods, which are known in themselves.

The peptides and their derivatives (variants, modified peptides, lipopeptides, multi-epitope fragments, fusion proteins) as defined above are prepared by conventional techniques known to those skilled in the art, in particular by solid-phase or liquid-phase synthesis or by expression of a recombinant DNA in an appropriate cell system (eukaryotic or prokaryotic).

More specifically:
the peptides and their derivatives (variants, multi-epitope peptides) can be solid-phase synthesized according to the Fmoc technique, originally described by Merrifield et al. (J. Am. Chem. Soc., 1965, 85: 2149-) and purified by reverse-phase high performance liquid chromatography,
the lipopeptides can in particular be prepared according to the method described in international applications WO 99/40113 or WO 99/51630,
the peptides and derivatives such as the variants, the multi-epitope fragments and the fusion proteins can also be produced from the corresponding cDNAs, obtained by any means known to those skilled in the art; the cDNA is cloned into a eukaryotic or prokaryotic expression vector and the protein or the fragment produced in the cells modified with the recombinant vector is purified by any appropriate means, in particular by affinity chromography.

In addition to the above arrangements, the invention also comprises other arrangements, which will emerge from the description which follows, which refers to examples of implementation of the subject of the present invention, with reference to the appended drawings in which:

FIG. 1 represents the peptide sequence of human midkine (SEQ ID NO: 2). The complete sequence corresponds to the precursor. The signal peptide is indicated in bold characters and underlined;

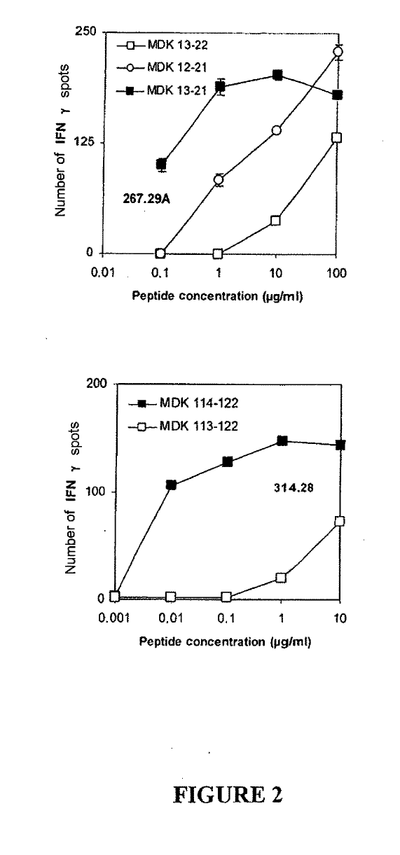

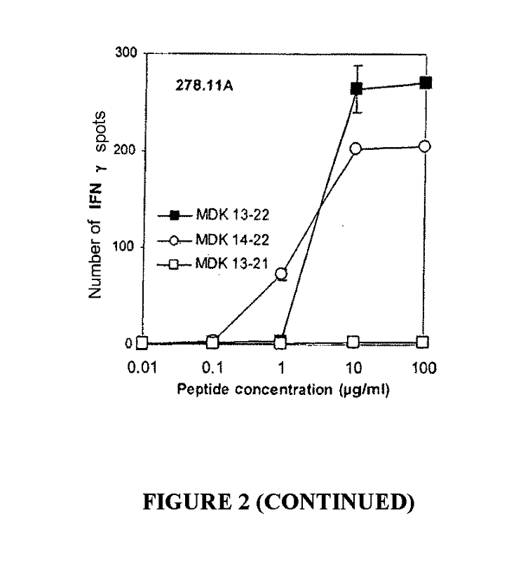

FIG. 2 illustrates the peptide specificity of the CD8⁺ T lymphocytes induced against the midkine peptides. The T lymphocyte lines (267.29A, 278.11A, 314.28) were obtained by stimulation of T lymphocytes from three healthy individuals expressing HLA-A2 (267, 278, 314). After four weeks of culture, their specificity was tested by IFN-γ Elispot;

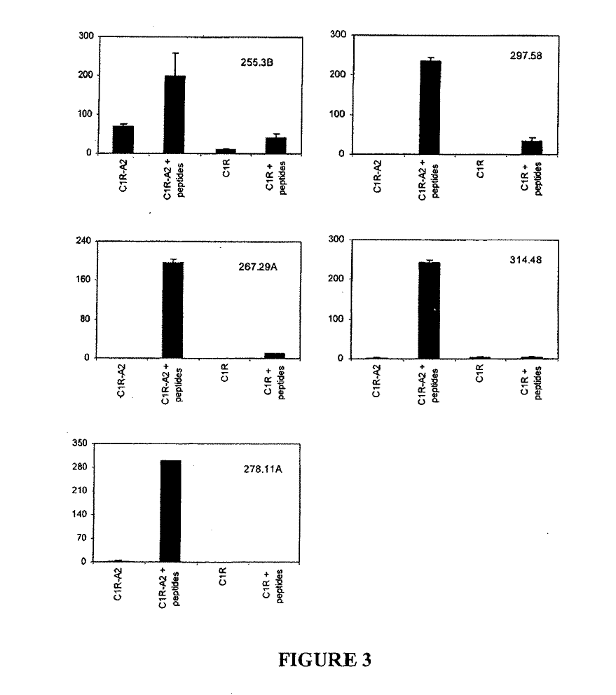

FIG. 3 illustrates the HLA-A2 restriction of the CD8⁺ T lymphocytes specific for the midkine peptides. The restriction was evaluated by IFN-γ Elispot, using C1R cells and C1R-A2 cells (C1R cells transfected with HLA-A2);

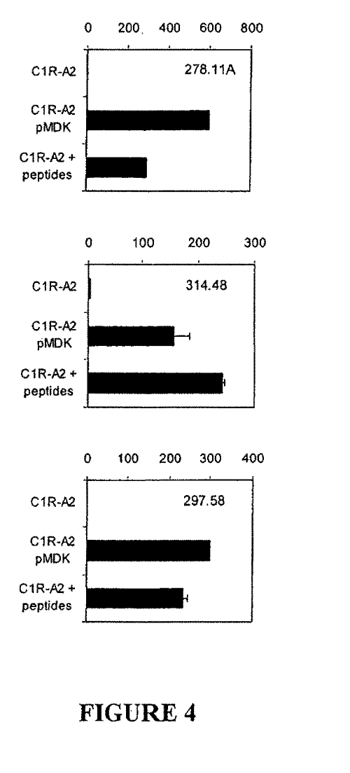

FIG. 4 illustrates the recognition of the cells transfected with a midkine expression plasmid, by CD8⁺ T lymphocytes specific for the midkine peptides. The C1R-A2 cells were transfected with a recombinant plasmid pcDNA 3.1 containing the coding sequence of midkine (pMDK). The activation of the CD8⁺ T lymphocytes by the pMDK-transfected C1R-A2 cells or the nontransfected cells was evaluated by IFN-γ Elispot;

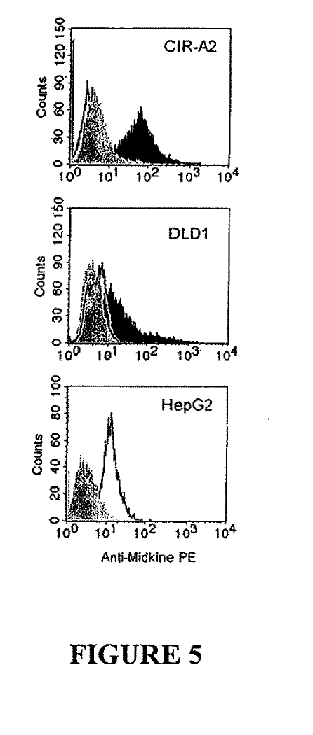

FIG. 5 illustrates the midkine expression in the tumor cells. The midkine expression was evaluated in C1R-A2, DLD-1 and Hep G2 cells by flow cytometry, using an anti-midkine antibody. Gray surface: negative control. Area under the black line: natural expression of midkine. Black surface: expression of midkine after transfection of the cells with a midkine expression plasmid;

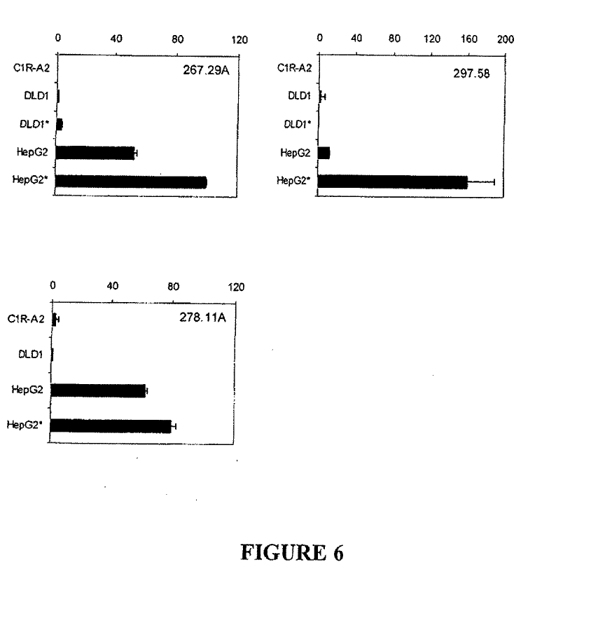

FIG. 6 illustrates the recognition of the tumor lines by the CD8⁺ T lymphocytes specific for the midkine peptides. The tumor recognition was tested by IFN-γ Elispot, using HLA-A2⁺ C1R-A2 (MDK), DLD-1 (MDK⁻) and Hep G2 (MDK⁺) cells. The cells marked with a star were cultured in the presence of IFN-γ;

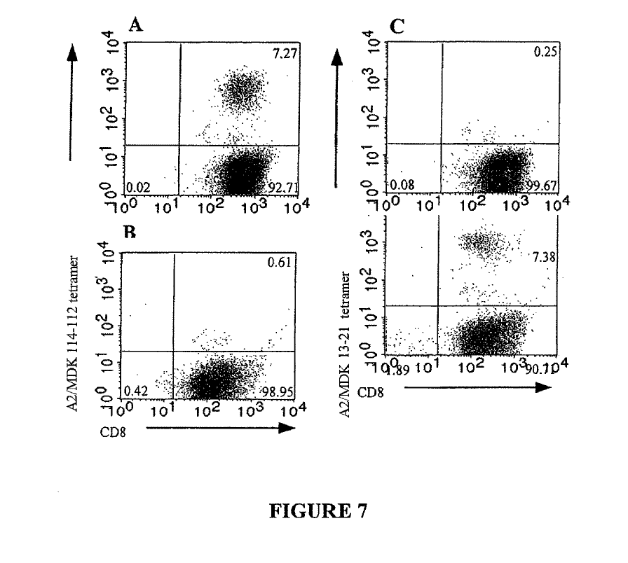

FIG. 7 illustrates the detection of midkine-specific CD8⁺ lymphocytes by labeling with specific tetramers. The T lymphocyte lines 314.7 (A and C) and 314.28 (B and D) are specific for the MDK 114-122 and MDK 13-21 peptides, respectively. Each line was labeled by means of an anti-CD8 antibody and the HLA-A2/MDK 114-112 (A and B) and HLA-A2/MDK 13-21 (C and D) tetramers, and analyzed by flow cytometry. The percentage of each population of cells is indicated in each quadrant;

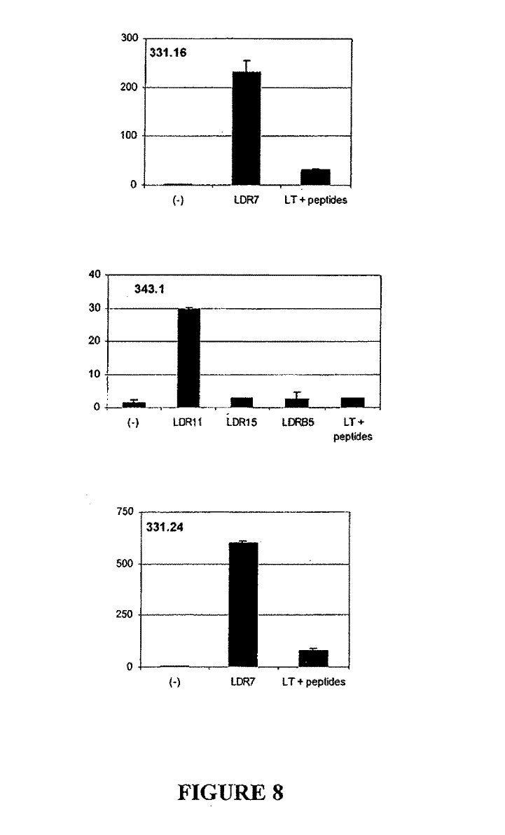

FIG. 8 illustrates the HLA II-restriction of the CD4⁺ T lymphocytes specific for midkine peptide 9-23. The restriction was evaluated by IFN-γ Elispot, using L cells transfected with an HLA II molecule (HLA-DR7, -DR11, -DR15, -DRB5) and loaded with peptide 9-23;

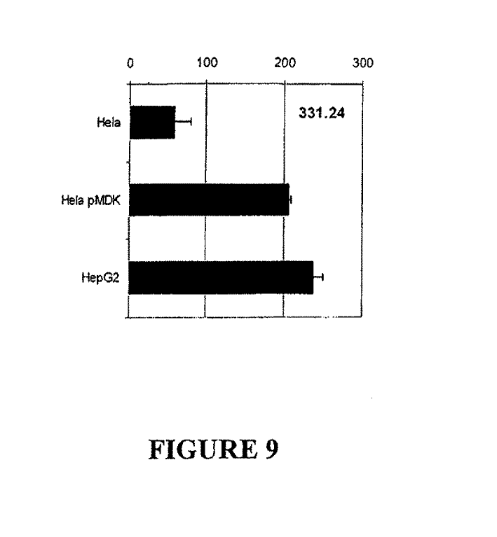

FIG. 9 illustrates the demonstration of the recognition of tumor lysates by the 331.24 T-line of CD4⁺ T lymphocytes specific for midkine peptide 9-23. The tumor recognition was tested by IFN-γ Elispot, using HeLa (MDK⁻), HeLa-pMDK (MDK⁺) and HepG2 (MDK⁺) cells.

### EXAMPLE 1

### Induction of a CD8⁺ T Response Specific for Peptides of the Midkine Protein

1) Materials and Methods

a) Peptides

Seven peptides representing potential CD8⁺ T epitopes restricted by the HLA-A2 molecule, which is the class I HLA allele most widely represented in the caucasian population, were selected using the BIMAS program (http://www-bimas.cit.nih.gov). The sequences of the peptides selected are shown in table IV and the appended sequence listing.

TABLE IV
List of selected peptides

| Peptide | Sequence | SEQ ID NO: |
|---|---|---|
| MDK13-21 | ALLALTSAV | 4 |
| MDK12-21 | LALLALTSAV | 3 |
| MDK14-22 | LLALTSAVA | 6 |
| MDK13-22 | ALLALTSAVA | 5 |
| MDK114-122 | AQCQETIRV | 8 |
| MDK113-122 | NAQCQETIRV | 7 |
| MDK 63-72 | AQTQRIRCRV | 17 |

The peptides were synthesized according to Fmoc strategy in solid-phase parallel synthesis, purified by HPLC and verified by mass spectroscopy (ES-MS).

b) Obtaining of HLA-A2-Restricted CD8⁺ T Lymphocyte Lines Specific for Midkine Peptides

The peripheral blood mononuclear cells (PBMCs) of healthy individuals possessing the HLA-A2 molecule were separated on a Ficoll gradient. The PBMCs were then cultured in AIM V medium (Life Technologies) and incubated overnight at 37° C. in the presence of 5% CO₂/95% air. The CD8⁺ T lymphocytes were purified from the nonadherent cells by immunomagnetic sorting, and frozen. The adherent cells were differentiated into immature dendritic cells by culturing for 5 days in AIM V medium containing 1000 U/ml of GM-CSF and 1000 U/ml of IL-4, and then into mature dendritic cells by culturing for 2 days in the presence of 1 μg/ml of LPS, 1000 U/ml of IL-4 and 1000 U/ml of GM-CSF. The mature dendritic cells were incubated in the presence of 5 μg/ml of beta-2-microglobulin and 10 μg/ml of each of the peptides of table IV. After 4 hours, the cells were washed and then placed in culture in 96-well plates, in the presence of purified CD8 T lymphocytes in IMDM medium containing 10% of group AB human serum, IL-6 (1000 U/ml) and IL-12 (5 ng/ml). Each week, the culture was restimulated with autologous mature dendritic cells loaded with the peptide mixture mentioned above, in medium containing 20 U/ml of IL-2 and 10 ng/ml of IL-7. After 4 weeks of culture, the specificity of the T cell lines contained in each well was tested by IFN-γ Elispot.

c) Presentation of the Midkine Protein to CD8⁺ T Lymphocytes Specific for the Midkine Peptides

The peptide-specific CD8⁺ T lymphocyte lines were cultured in the presence of C1R-A2 cells transfected with a recombinant plasmid pcDNA3.1 (Invitrogen) comprising the midkine coding sequence under the control of the CMV promoter and of the bovine growth hormone polyadenylation signal. The activation of the CD8⁺ T lymphocytes by these transfected C1R-A2 cells was evaluated by Elispot as specified below.

d) Recognition of Tumor Cells by the CD8⁺ T Lymphocytes Specific for the Midkine Peptides

The peptide-specific CD8⁺ T lymphocyte lines were cultured in the presence of various tumor lines: DLD-1 (ATCC® # CCL-221) and Hep G2 (ATCC® # HB-8065). The activation of the CD8⁺ T lymphocytes by these tumor cells was evaluated by Elispot as specified below.

e) Elispot

Anti-IFN-γ antibodies (1-D1K, Mabtech) diluted to 2.5 μg/ml in PBS buffer were adsorbed onto nitrocellulose plates (Millipore) for 1 hour at 37° C. The plates were then washed with PBS and then saturated with Iscove medium containing 10% of group AB human serum (100 μg/well), for 2 h at 37° C.

The antigen-presenting cells are either cells of the lymphoblastoid B cell line C1R (Hogan et al., J. Immunol., 1988, 141, 2519-2525), devoid of HLA-A and HLA-B molecules, transfected with the cDNA encoding HLA-A2 (C1R-A2) and loaded with a single peptide (10 μg of peptide) or the mixture of peptides (10 μg of each peptide), or C1R-A2 cells transfected with a midkine expression plasmid, or else tumor cells expressing midkine.

In order to verify the specificity of the lines with respect to the HLA-A2 molecule, the C1R cells transfected with HLA-A2 (30 000 cells/well) and 5000 test lymphocytes were then added to the plates and incubated for 24 h at 37° C., in the presence or absence of a single peptide (10 μg of peptide) or of a mixture of peptides (10 μg of each peptide). For the dose-response analyses, the peptides are used at various concentrations ranging from 0.001 to 10 μg/ml.

In order to analyze the recognition of the midkine-transfected cells expressing HLA-A2, by the peptide-specific CD8⁺ T lymphocytes, the C1R cells transfected with HLA-A2 and with a midkine expression plasmid (30 000 cells/well) and 5000 test lymphocytes were then added to the plates and incubated for 24 h at 37° C.

In order to analyze the recognition of the tumor cells expressing midkine, by the peptide-specific CD8⁺ T lymphocytes, the tumor cells (30 000 cells/well) and 5000 test lymphocytes were then added to the plates and incubated for 24 h at 37° C.

After three successive washes with water, PBS buffer/0.05% Tween and PBS alone, 100 μl of biotinylated anti-IFN-γ secondary antibody (7-B6-1-biotin, Mabtech), diluted to 0.25 μg/ml in PBS containing 1% BSA, were added to each well. After one hour of incubation at ambient temperature, the plates were washed again and then incubated for one hour at ambient temperature with 100 μg/well of Extravidin-AKP (E-2636, Sigma), diluted to 1/6000. After washing of the plates in PBS buffer, 100 μl of NBT/BCIP substrate B-5655, Sigma), diluted in water (1 tablet in 10 ml of water), were distributed in each well. The immunoenzymatic visualization was stopped after approximately 10 minutes, by thorough rinsing of the plates in water. After drying of the plates, the colored spots were counted using an automatic reader (AID). The lines are considered to be positive when the number of spots is more than three times that obtained with the negative control (control without peptides) with a minimum of 50 spots. The control without presenting cells makes it possible to verify the specificity of the response for HLA-A2 (restriction control).

2) Results

The ability of the midkine protein to induce a tumor-cell-specific cellular immune response was evaluated. To do this, CD8⁺ T epitopes restricted by the HLA-A2 molecule, which is the HLA I molecule most frequent in the caucasian population, were first of all identified in the midkine sequence. Next, the ability of the CD8⁺ T cells induced by these epitopes to selectively recognize a tumor line expressing midkine was analyzed.

The peptides synthesized were tested for their ability to induce an in vitro response using cells collected from healthy individuals who possess the HLA-A2 molecule. Six of these peptides induced CD8⁺ T lymphocytes: MDK 13-21, MDK 13-22, MDK 12-21, MDK 14-22, MDK 113-122 and MDK 114-122. As shown in FIG. 2, the CD8⁺ T lymphocyte line 267.29A is specific for the peptides 12-21, 13-21 and 13-22. The 278.11A line is specific for the peptides 13-21, 13-22 and 14-22. The 314.28 line is specific for peptide 114-122 and, to a lesser extent, for the peptide 113-122. The peptides 12-21, 13-21, 13-22, 14-22, 113-122 and 114-122 are therefore immunogenic and induce CD8⁺ T lymphocytes in healthy HLA-A2⁺ donors.

The HLA-A2 restriction of the peptide-specific CD8⁺ T lymphocyte lines is shown in FIG. 3. Only the HLA-A2 (C1R-A2) cells can present the peptides to the specific T lymphocyte lines. The C1R (HLA-A2⁻) cells do not stimulate them, even in the presence of the peptides.

In order to verify that the presenting cells were capable of correctly processing midkine, the C1R-A2 cells were transfected with a recombinant plasmid pcDNA3.1 comprising the midkine coding sequence. FIG. 4 shows that the CD8⁺ T lymphocyte lines 278.11A (specific for the peptides 13-22 and 14-22), 297.58 (specific for the peptides 12-21, 13-21 and 14-22) and 314.48 (specific for the peptide 114-122) are activated by the transfected cells and by the C1R-A2 cells loaded with the peptides, but not by the nontransfected cells.

The recognition of tumor lines expressing or not expressing midkine, by CD8⁺ T lymphocytes specific for midkine peptides, was also studied. FIG. 6 shows the expression or non-expression of midkine by the various lines. In FIG. 7, it is observed that the CD8⁺ T lymphocyte lines 267.29A (specific for the peptides 13-22, 12-22 and 13-21), 278.11A (specific for the peptides 13-22 and 14-22) and 314.48 (specific for the peptide 114-122) recognize Hep G2 cells which naturally expressed midkine, but not C1R-A2 and DLD-1 cells which do not express midkine. The recognition is slightly better when the Hep G2 cells are cultured in the presence of IFN-γ, owing to the increased expression of HLA molecules on the cells. The 297.58 line (specific for the peptides 12-21, 13-21 and 14-22) recognizes the Hep G2 cells only when they are cultured in the presence of IFN-γ.

All these results show that midkine contains six peptides divided up into two groups of overlapping peptides capable of inducing activation of HLA-A2-restricted CD8% T lymphocytes which selectively recognize tumor cells expressing midkine.

### EXAMPLE 2

### Detection of CD8⁺ T Lymphocytes Specific for Midkine Peptides by Labeling with Specific Tetramers

Each lymphocyte line (500 000 cells) obtained in example 1 was labeled for 1 hour, in the dark and at 4° C., with 50 μg/ml of tetramer in 200 μl of PBS/2% FCS. These tetramers are biotinylated HLA-A2 molecules loaded with the peptide 13-21 or 114-122 and complexed with phycoerythrin-labeled streptavidin, and prepared according to the technique described in Novak et al. (J. Clin. Investig., 1999, 104, R63-R67) or in Kuroda et al. (J. Virol., 2000, 74, 18, 8751-8756). The cells were then washed twice in PBS and then labeled for 30 min at 4° C. using an FITC anti-CD8 antibody (BD Biosciences). After washing in PBS, the cells were fixed with 50 μl of PBS containing 1% paraformaldehyde (PAF). The labelings were analyzed on a FACSCalibur flow cytometer (BD Biosciences). The results are shown in FIG. 7.

### EXAMPLE 3

### Induction of a CD4⁺ T Response Specific for Peptides of the Midkine Protein

1) Materials and Methods

a) Peptides

Peptides of 15 amino acids (15-mers) covering the entire sequence of human midkine (SwissProt P21741, SEQ ID NO: 2 and FIG. 1) were selected according to the presence of aromatic or hydrophobic residues at position 3 or 4, for anchoring in the P1 pocket of HLA-DR and HLA-DP4 molecules.

The sequences of the peptides selected are given in table V and the appended sequence listing.

The peptides were synthesized according to the Fmoc strategy in solid-phase parallel synthesis, purified by HPLC and verified by mass spectrometry (ES-MS).

TABLE V
Peptides selected (SEQ ID NO: 9, 10,
13-15 and 18-30)

| Peptide | Positions* | Sequence |
|---|---|---|
| MDK1 | MDK 1-15 | M Q H R G F L L L T L L A L L |
| MDK2 | MDK 4-18 | R G F L L L T L L A L L A L T |
| MDK3 | MDK 9-23 | L T L L A L L A L T S A V A K |
| MDK4 | MDK 14-28 | L L A L T S A V A K K K D K V |
| MDK5 | MDK 18-32 | T S A V A K K K D K V K K G G |
| MDK6 | MDK 25-39 | K D K V K K G G P G S E C A E |
| MDK7 | MDK 38-52 | A E W A W G P C T P S S K D C |
| MDK8 | MDK 52-64 | C G V G F R E G T C G A Q T Q |
| MDK9 | MDK 64-78 | Q T Q R I R C R V P C N W K K |
| MDK10 | MDK 70-84 | C R V P C N W K K E F G A D C |
| MDK11 | MDK 74-88 | C N W K K E F G A D C K Y K F |
| MDK12 | MDK 78-92 | K E F G A D C K Y K F E N W G |
| MDK13 | MDK 84-98 | C K Y K F E N W G A C D G G T |
| MDK14 | MDK 89-103 | E N W G A C D G G T G T K V R |
| MDK15 | MDK 99-113 | G T K V R Q G T L K K A R Y N |
| MDK16 | MDK 105-119 | G T L K K A R Y N A Q C Q E T |
| MDK17 | MDK 110-124 | A R Y N A Q C Q E T I R V T K |
| MDK18 | MDK 119-133 | E T I R V T K P C T P K T K A |

*The positions are numbered with reference to the sequence of the human midkine precursor of 143 amino acids (SwissProt P21741, FIG. 1 and SEQ ID NO: 2).

b) HLA II/Peptide Binding Assay

The assays for binding to HLA II molecules are competition binding assays with immunoenzymatic visualization, as described in U.S. Pat. No. 6,649,166 and PCT international application WO 03/040299, respectively, for the HLA-DR and HLA-DP4 molecules. The implementation of these assays for measuring the binding activity of peptides derived from various antigens is illustrated in U.S. Pat. No. 6,649,166 and PCT international applications WO 02/090382, WO 03/040299 and WO 2004/014936.

More specifically, the peptides: HA 306-318 (PKYVKQNTLKLAT, SEQ ID NO: 31), A3 152-166 (EAEQLRAYLDGTGVE, SEQ ID NO: 32), MT 2-16 (AKTIAYDEEARRGLE, SEQ ID NO: 33), B1 21-36 (TERVRLVTRHIYNREE, SEQ ID NO: 34) YKL (AAYAAAKAAALAA, SEQ ID NO: 35), LOL 191-210 (ESWGAVWRIDTPDKLTGPFT, SEQ ID NO: 36) Oxy 271-287 (EKKYFAATQFEPLAARL, SEQ ID NO: 37) and E2/E168 (AGDLLAIETDKATI SEQ ID NO: 38), biotinylated at the NH₂-terminal residue, according to the protocol described in Texier et al., J. Immunol., 2000, 164, 3177-3184, are used as a tracer under the conditions as specified in the table below.

TABLE VI
Conditions of the test for binding to HLA II molecules

| | | | Tracer | | Incu- | |
|---|---|---|---|---|---|---|
| | | | concen- | | bation | |
| | HLA II | | tration | Optimal | time | IC₅₀ |
| Alleles | dilution | Tracers | (nM) | pH | (h) | (nM) |
| DRB1*0101 | 1/40 | HA 306-318 | 1 | 6 | 24 | 2 |
| DRB1*0301 | 1/20 | MT 2-16 | 100 | 4.5 | 72 | 239 |
| DRB1*0401 | 1/60 | HA 306-318 | 10 | 6 | 24 | 6 |
| DRB1*0701 | 1/80 | YKL | 10 | 5 | 24 | 4 |
| DRB1*1101 | 1/80 | HA 306-318 | 10 | 5 | 24 | 9 |
| DRB1*1301 | 1/40 | B1 21-36 | 100 | 4.5 | 72 | 39 |
| DRB1*1501 | 1/100 | A3 152-166 | 30 | 4.5 | 72 | 19 |
| DRB4*0101 | 1/30 | E2/E168 | 10 | 5 | 72 | 3 |
| DRB5*0101 | 1/80 | HA 306-318 | 10 | 5.5 | 24 | 5 |
| DRB3*0101 | 1/40 | Lol 191-120 | 20 | 5.5 | 24 | 21 |
| DBP1*0401 | 1/100 | Oxy 271-287 | 10 | 5 | 24 | 11 |
| DPB1*0402 | 1/40 | Oxy 271-287 | 10 | 5 | 24 | 10 |

The sensitivity of each test is reflected by the IC₅₀ values observed with the nonbiotinylated peptides which correspond to the tracers. The concentration (nM) of competitor peptide which inhibits 50% of the maximum binding of the biotinylated tracer peptide (IC₅₀) was calculated for each peptide. The results are expressed in the form of relative activity (ratio of the IC₅₀ of the competitor peptide to that of the reference peptide (nonbiotinylated peptide which corresponds to the tracer)). A relative activity of less than 100 characterizes the active peptides.

c) Obtaining of CD4⁺ T Lymphocyte Lines Specific for Midkine Peptides and Restricted by the Predominant HLA II Molecules

The peripheral blood mononuclear cells (PBMCs) of healthy individuals, of whom the HLA-DR and HLA-DP genotype was determined beforehand by SSP, using the Olerup SSP™ HLA-DPB1 and HLA-DRB1 kit, was separated on a Ficoll gradient. The PBMCs were then cultured in AIM V medium (Life Technologies) and incubated in flasks, in an incubator at 37° C. in the presence of 5% CO₂/95% air. After overnight incubation, the nonadherent cells were recovered, and then the CD4⁺ T lymphocytes were purified using anti-CD4 antibodies coupled to magnetic beads (Miltenyi Biotec kit), and frozen. The adherent cells were incubated for 5 days in AIM V medium containing 1000 U/ml of GM-CSF and 1000 U/ml of IL-4, and then the cells that had differentiated into dendritic cells (immature dendritic cells) were subsequently cultured for 2 days, in the presence of 1 μg/ml of LPS, 1000 U/ml of IL-4 and 1000 U/ml of GM-CSF, so as to induce maturation thereof.

The mature dendritic cells (100 000 cells/well) were then incubated with a mixture of peptides (10 μg of each peptide in IMDN medium (Invitrogen) supplemented with glutamine (24 mM, Sigma), asparagine (55 mM, Sigma), arginine (150 mM, Sigma), penicillin (500 IU/ml, Invitrogen), streptomycin (50 mg/ml, Invitrogen) and 10% of human serum)), for 4 hours at 37° C. The mature dendritic cells were subsequently washed and then incubated, in the presence of the CD4⁺ T lymphocytes (100 000 cells/well) thawed beforehand, in medium containing 1000 U/ml of IL-6 and 10 ng/ml of IL-12. After 7 days (D7), the culture was stimulated a first time by means of mature dendritic cells previously thawed and loaded with two mixtures of peptides covering the entire midkine sequence (mixture of peptides MDK1 to MDK9 and then mixture of peptides MDK 1 to MDK 18), in medium containing IL-2 (10 U/ml) and IL-7 (5 mg/ml). After three further simulations (D14, D21, D28) by means of loaded dendritic cells, in medium containing only IL-7 (5 ng/ml), the cells were tested by Elispot, at least 6 days after the final stimulation.

d) Elispot

Anti-IFN-γ antibodies (1-D1K, Mabtech), diluted to 2.5 μg/ml in PBS buffer, were adsorbed onto nitrocellulose plates (Millipore) for 1 hour at 37° C. The plates were then washed with PBS and then saturated with Iscove medium containing 10% of group AB human serum (100 μg/well), for 2 h at 37° C. The antigen-presenting cells are either immature autologous dendritic cells prepared as specified above, or a line of mice fibroblasts (L line), transfected with the cDNA encoding one of the HLA-DR or HLA-DP4 molecules to be tested (Yu et al., Hum. Immunol., 1990, 27, 132-135), so as to verify the specificity of the lines with respect to the HLA-D and HLA-DP4 molecules. The dendritic cells (10⁵ cells/well) or L cells transfected with one of the HLA-DR or HLA-DP4 molecules (30 000 cells/well) and 5000 test lymphocytes were then added to the plates and incubated for 24 h at 37° C., in the presence or absence of a single peptide (10 μg) or of a mixture of peptides (10 μg of each peptide). After three successive washes with water, PBS buffer/0.05% Tween and PBS alone, 100 μl of biotin-conjugated anti-IFN-γ secondary antibody (7-B6-1-biotin, Mabtech), diluted to 0.25 μg/ml in PBS containing 1% BSA, were added to each well. After one hour of incubation, the plates were washed again and incubated with 100 μl/well of Extravidin-AKP (E-2636, Sigma), diluted to 1/6000. After washing of the plates in PBS buffer, 100 μl of NBT/BCIP substrate (B-5655, Sigma), diluted in water (1 tablet in 10 ml of water), were distributed in each well. The immunoenzymatic visualization was stopped after approximately 10 minutes, by thorough rinsing of the plates in water, and the colored spots were counted using an automatic reader (AID). The lines are considered to be positive when the number of spots is more than three times that obtained with the negative control (control without peptides) with a minimum of 50 spots. The control without presenting cells makes it possible to verify the specificity of the response for HLA-DR or HLA-DP4 (restriction control).

e) Recognition of Tumor Cells by the CD4⁺ T Lymphocytes Specific for the Midkine Peptides

The tumor lines tested are the Hep G2 line which expresses midkine, the HeLa tumor line which does not express midkine and the HeLa-pMDK line which corresponds to HeLa cells transiently transfected with a midkine expression plasmid as described in example 1. The collected cells were lysed by means of freezing/thawing cycles. The 331.24 line of CD4⁺ T lymphocytes specific for the midkine peptide 9-23 was incubated in the presence of dendritic cells pre-loaded with the tumor line lysates, and its activation was evaluated by Elispot as specified above.

2) Results

a) Binding Activity of the Midkine Peptides with Respect to HLA II Molecules

Most of the sites for binding to class II HLA molecules are located in the N-terminal portion of midkine, i.e. in the signal peptide (1-22; table VII).

TABLE VII
Relative binding * activities of the midkine peptides with respect to the 12 predominant HLA II molecules

| peptides | DR1 | DR3 | DR4 | DR7 | DR11 | DR13 | DR15 | DRB3 | DRB4 | DRB5 | DP401 | DP402 | Total |
|---|---|---|---|---|---|---|---|---|---|---|---|---|---|
| MDK | 21 | >419 | 226 | 49 | 7 | >2 537 | 211 | 267 | 204 | 161 | 20 | 19 | 5 |
| 1-15 | | | | | | | | | | | | | |
| MDK | 21 | >419 | 136 | 20 | 94 | >2 537 | 19 | 37 | 65 | 46 | 6 | 18 | 9 |
| 4-18 | | | | | | | | | | | | | |
| MDK | 0.2 | >419 | 1 | 13 | 0.3 | >2 537 | 5 | >485 | >28 868 | 2 | 94 | 29 | 8 |
| 9-23 | | | | | | | | | | | | | |
| MDK | 34 | >419 | 401 | 590 | 48 | 45 | >529 | >485 | >28 868 | 0.1 | >879 | >976 | 4 |
| 14-28 | | | | | | | | | | | | | |
| MDK | >5 291 | >419 | >1 812 | >2 479 | >1 086 | 132 | >529 | >485 | >28 868 | >2 100 | >879 | >976 | 0 |
| 18-32 | | | | | | | | | | | | | |
| MDK | 1 251 | >419 | >1 812 | >2 479 | >1 086 | >2 537 | >529 | >485 | >28 868 | >2 100 | >879 | >976 | 0 |
| 25-39 | | | | | | | | | | | | | |
| MDK | 1 305 | >419 | 1 859 | >2 479 | 923 | >2 537 | >529 | >485 | >28 868 | >2 100 | >879 | 239 | 0 |
| 38-52 | | | | | | | | | | | | | |
| MDK | 32 | >419 | 701 | >2 479 | 833 | >2 537 | >529 | >485 | >28 868 | >2 100 | >879 | >976 | 1 |
| 52-64 | | | | | | | | | | | | | |
| MDK | 246 | >419 | 558 | 2 066 | 504 | >2 537 | >529 | >485 | 1 155 | 61 | >879 | >976 | 1 |
| 64-78 | | | | | | | | | | | | | |
| MDK | 53 | >419 | 1 562 | >2 479 | >1 086 | >2 537 | >529 | 621 | >28 868 | >2 100 | 7 | >976 | 2 |
| 70-84 | | | | | | | | | | | | | |
| MDK | 333 | 2 | >1 812 | >2 479 | 1 231 | >2 537 | >529 | 877 | >28 868 | 714 | >879 | >976 | 1 |
| 74-88 | | | | | | | | | | | | | |
| MDK | 299 | 1 | 457 | >2 479 | 800 | >2 537 | 216 | 226 | >28 868 | 114 | 167 | 378 | 1 |
| 78-92 | | | | | | | | | | | | | |
| MDK | 187 | >419 | 362 | >2 479 | >1 086 | >2 537 | 141 | >485 | >28 868 | 292 | 52 | 49 | 2 |
| 84-98 | | | | | | | | | | | | | |
| MDK | 1 460 | >419 | >1 812 | >2 479 | >1 086 | >2 537 | >529 | 2 333 | >28 868 | >2 100 | >879 | >976 | 0 |
| 89-103 | | | | | | | | | | | | | |
| MDK | 3 000 | >419 | >1 812 | >2 479 | >1 086 | 74 | >529 | >485 | >28 868 | 215 | >879 | >976 | 1 |
| 99-113 | | | | | | | | | | | | | |
| MDK | 97 | >419 | 492 | >2 479 | 1 008 | >2 537 | 225 | >485 | >28 868 | >2 100 | >879 | >976 | 1 |
| 105-119 | | | | | | | | | | | | | |
| MDK | 10 | >419 | 6 | 158 | 69 | >2 537 | >529 | >485 | >28 868 | 15 | >879 | >976 | 4 |
| 110-124 | | | | | | | | | | | | | |
| MDK | 2 | >419 | 1 289 | 819 | 763 | >2 537 | >529 | >485 | >28 868 | 26 | >879 | >976 | 2 |
| 119-133 | | | | | | | | | | | | | |

* The values are the means of at least two independent experiments.

The peptides of the N-terminal region have good affinity for at least 4 HLA II molecules. In particular, the peptide 9-23 binds to 8 different HLA II molecules with relative affinities that often reflect a high affinity (relative activity less than 10). Other peptides also bind to several HLA II molecules, such as the peptides 1-15, 4-18 and 14-28.

On the other hand, the peptides derived from the rest of the sequence do not exhibit any significant binding activity for at least four HLA II molecules predominant in the caucasian population, with the exception of a peptide of the C-terminal region (110-124) which binds with good affinity to four HLA II molecules.

b) Induction of a Specific CD4⁺ T Response by the Midkine Peptides

The ability of the midkine peptides to induce, in vitro, a stimulation of specific CD4⁺ T lymphocytes was evaluated using blood samples from healthy individuals (individuals with no tumor). It involved evaluating the ability to recruit CD4⁺ precursor lymphocytes although they are present at a very low frequency in a naïve individual, i.e. to perform an in vitro immunization by means of these peptides.

The CD4⁺ T lymphocyte lines 331.16, 331.24 and 343.1 were obtained by in vitro stimulation of T lymphocytes by means of mature autologous dendritic cells loaded with two pools of peptides covering the entire midkine sequence. The study of their specificity was carried out by IFN-γ Elispot and showed that the three lines were specific for the peptide 9-23. Each line was tested, by IFN-γ Elispot, for its ability to be stimulated by L cells transfected with an HLA-DR or HLA-DP4 molecule and loaded with the peptide 9-23. FIG. 8 shows that the peptide 9-23 can be presented by the DR7 molecule to the lines of donor 331 (331.16 and 331.24) and that the 343.1 line is DR11-restricted but not DR15- and DRB5-restricted.

The CD4⁺ T lymphocyte line 331.24 was incubated in the presence of dendritic cells pre-loaded with the tumor line lysates and its activation was evaluated by IFN-γ Elispot. FIG. 9 shows that the 331.24 line is stimulated by dendritic cells loaded with the lysate of transfected HeLa cells, but not by the nontransfected HeLa cells. This confirms the specificity of the T lymphocyte line 331.24 and its ability to recognize midkine present in the lysate of transfected cells. It also recognizes the midkine naturally produced by the Hep G2 tumor line.

All the results show that the peptide 9-23 binds to 8 different HLA II molecules and induces a specific CD4⁺ T response, in vitro, which is restricted by different class II HLA molecules. Furthermore, the CD4⁺ T cells induced against this peptide can recognize lysates of tumors expressing midkine and presented by dendritic cells. Since this peptide overlaps with the signal peptide (1-22), it can be deduced from these experiments that the peptide 9-22 also comprises CD4⁺ T epitopes since the midkine signal peptide is cleaved, in the cell, between amino acids 22 and 23. It is interesting to note that the peptides 9-23 and 9-22 include the peptides 12-21, 13-21, 13-22 and 14-22 which comprise CD8⁺ T epitopes. The peptides 9-23 and 9-22 can therefore induce CD4⁺ T and CD8⁺ T responses specific for tumors expressing midkine.

As emerges from the above, the invention is not in any way limited to those of its methods of implementation, execution and application that have just been more explicitly described; on the contrary, it encompasses all the variants thereof that may occur to a person skilled in the art, without departing from either the context or the scope of the present invention.

1. An isolated peptide derived from the midkine protein, comprising at least one CD4⁺ T or CD8⁺ T epitope restricted by the HLA molecules predominant in the caucasian population.

2. The isolated peptide as claimed in claim 1, characterized in that said peptide consists of the human midkine protein of sequence SEQ ID NO: 2.

3. The isolated peptide as claimed in claim 1, characterized in that said peptide is a fragment of at least 8 amino acids of the midkine protein, comprising at least one HLA-A2 molecule-restricted CD8⁺ T epitope, said peptide comprising at least positions 14 to 21 or 114 to 122 of the amino acid sequence of said midkine protein.

4. The isolated peptide as claimed in claim 3, characterized in that said peptide consists of positions 12 to 21, 13 to 21, 13 to 22, 14 to 22, 113 to 122 or 114 to 122 of the amino acid sequence of the midkine protein.

5. The isolated peptide as claimed in claim 1, characterized in that said peptide is a fragment of at least 8 amino acids of midkine, comprising at least one CD4⁺ T epitope restricted by at least four different HLA II molecules predominant in the caucasian population, said peptide comprising at least positions 9 to 15, 14 to 28 or 110 to 124 of the amino acid sequence of said midkine protein.

6. The isolated peptide as claimed in claim 5, characterized in that said peptide consists of positions 1 to 15, 4 to 18 or 14 to 28 of the amino acid sequence of said midkine protein.

7. The isolated peptide as claimed in claim 1, characterized in that said peptide comprises at least one CD8⁺ T epitope restricted by the HLA-A2 molecule and at least one CD4⁺ T epitope restricted by at least four different HLA II molecules predominant in the caucasian population, said peptide consisting of positions 9 to 21, 9 to 22, 9 to 23 or 110 to 124 of the amino acid sequence of said midkine protein.

8. The isolated peptide as claimed in claim 1, characterized in that said peptide is a multi-epitope peptide comprising the concatenation of at least two identical or different epitopes, at least one of which is a midkine CD4⁺ T and/or CD8⁺ T epitope as defined in claim 1.

9. The isolated peptide as claimed in claim 8, characterized in that said multi-epitope peptide comprises a CD4⁺ T or CD8⁺ T epitope of another tumor antigen.

10. The isolated peptide as claimed in claim 1, characterized in that said peptide is fused to a heterologous protein or protein fragment.

11. The isolated peptide as claimed in claim 1, characterized in that said peptide is a lipopeptide.

12. An isolated polynucleotide encoding a peptide as defined in claim 1.

13. The isolated polynucleotide as claimed in claim 12, characterized in that said polynucleotide is inserted into an expression vector.

14. An immunogenic composition which comprises an amount of the isolated peptide of claim 1 effective to treat cancer and a pharmaceutically acceptable vehicle, a carrier substance and/or an adjuvant.

15. (canceled)

16. (canceled)

17. The immunogenic composition as claimed in claim 14, characterized in that the cancer is selected from the group consisting of: esophageal, stomach, colon, pancreatic, thyroid, lung, breast, bladder, uterine, ovarian and prostrate cancers, hepatocellular carcinomas, osteosarcomas, neuroblastomas, glioblastomas, astrocytomas, leukemias and Wilms tumors.

18. A vaccine composition, characterized in that it comprises at least one isolated peptide as defined in claim 3, and a pharmaceutically acceptable vehicle, a carrier substance or an adjuvant.

19. An in vitro method for immunomonitoring of the cellular response against midkine in an individual with a cancer, characterized in that it comprises:
bringing a biological sample from said individual into contact with a peptide as defined in claim 1, and
detecting midkine-specific CD4⁺ T and/or CD8⁺ T lymphocytes by any appropriate means.

20. A kit for immunomonitoring of the cellular response against midkine, characterized in that it comprises a peptide as defined in claim 1.

21. (canceled)

22. A polynucleotide encoding the peptide as claimed in claim 3.

23. An expression vector comprising the polynucleotide as claimed in claim 22.

24. A host cell modified with the polynucleotide as claimed in claim 22 or the vector as claimed in claim 23.

25. A method for preventing or treating a cancer associated with tumor overexpression of the midkine protein in an individual, comprising the administration of a vaccine composition comprising a peptide derived from the midkine protein as claimed in claim 3 or an expression vector encoding said peptide, and a pharmaceutically acceptable vehicle, a carrier substance or an adjuvant.

26. A method for preventing or treating a cancer associated with tumor overexpression of the midkine protein in an individual, comprising the administration of a vaccine composition comprising a peptide derived from the midkine protein as claimed in claim 5 or an expression vector encoding said peptide, and a pharmaceutically acceptable vehicle, a carrier substance or an adjuvant.

27. A method for preventing or treating a cancer associated with tumor overexpression of the midkine protein in an individual, comprising the administration of a vaccine composition comprising a peptide derived from the midkine protein as claimed in claim 7 or an expression vector encoding said peptide, and a pharmaceutically acceptable vehicle, a carrier substance or an adjuvant.
